(19) United States
(12) Patent Application Publication
(10) Pub. No.: US 2026/0083749 A1
(43) Pub. Date: Mar. 26, 2026

(54) ANTI-CD123 IMMUNOCONJUGATES FOR THE TREATMENT OF ACUTE MYELOID LEUKEMIA

(71) Applicant: ImmunoGen, Inc., North Chicago, IL (US)

(72) Inventor: Patrick Zweidler-McKay, North Chicago, IL (US)

(21) Appl. No.: 19/204,211

(22) Filed: May 9, 2025

Related U.S. Application Data
(60) Provisional application No. 63/645,620, filed on May 10, 2024.

(51) Int. Cl. A61K 31/5513; A61K 31/575; A61K 31/7068; A61K 35/28; A61K 47/68; A61P 35/02

(52) U.S. Cl. CPC A61K 31/5513; A61K 31/575; A61K 31/7068; A61K 35/28; A61K 47/6845; A61P 35/02

(57) ABSTRACT

Methods and uses of immunoconjugates that bind to CD123 (e.g., pivekimab sunirine) in patients with acute myeloid leukemia (AML) are provided. Such immunoconjugates can be used as monotherapies or can be used in combination with BCL-2 inhibitors (e.g., venetoclax), and/or hypomethylating agents (e.g., azacitidine or decitabine) to prepare patients with AML for hematopoietic stem cell transplant and/or to achieve complete remissions in patients with AML, including those with poor prognostic markers.

### CROSS-REFERENCE TO RELATED PATENT APPLICATIONS

This patent application claims the benefit of U.S. Provisional Patent Application No. 63/645,620, filed May 10, 2024. Each of the aforementioned patent applications is hereby incorporated by reference in its entirety.

### FIELD OF THE DISCLOSURE

The field of the invention generally relates to methods of treating acute myeloid leukemia (AML), including newly diagnosed and relapsed/refractory AML, in patients and methods for preparing patients with AML for stem cell transplants using anti-CD123 immunoconjugates, e.g., pivekimab sunirine. In some aspects, a hypomethylating agent (HMA) and/or a B-cell leukemia/lymphoma-2 (BCL-2) antagonist can be used in combination with the anti-CD123 immunoconjugates.

### REFERENCE TO SEQUENCE LISTING SUBMITTED ELECTRONICALLY

The content of the electronically submitted sequence listing (Name: 6776-7001_Sequence-Listing.xml; Size: 10,397 bytes; and Date of Creation: May 8, 2025) filed with the application is incorporated herein by reference in its entirety.

### BACKGROUND

Acute myeloid leukemia (AML) is the most common form of acute leukemia among adults and accounts for the largest number of deaths from leukemias in the United States. As of 2017, an estimated 21,380 people will be diagnosed with AML per year and 10,590 patients will die of the disease (Siegel et al., CA Cancer J Clin. 2017; 67(1):7-30 (2017)). The median age of diagnosis is 66 years. Frontline chemotherapy in AML is reported to induce a complete response (CR) in 70%-80% of patients who are 60 years of age or younger and in approximately 50% of older patients. “Fit” patients are judged to be able to tolerate intensive treatment, are often younger (<60 years), and typically receive one to two cycles of induction with “7+3,” a combination of cytarabine and anthracycline, typically daunorubicin. Following this, these fit patients may receive high-dose cytarabine for one or more cycles and may receive a stem cell transplant. Standard induction and post-induction therapies result in a median duration of remission of approximately one year and potential cures in 25%-35% of the patients. “Unfit” patients, often older, typically receive venetoclax and azacitidine, a hypomethylating agent. The majority of AML patients will eventually relapse, and AML salvage regimens offer poor outcomes with significant toxicity. Thus, novel therapies with limited toxicity are needed.

CD123 is the alpha-subunit of the interleukin-3 receptor (IL-3Ra). CD123 expression is low on normal hematopoietic stem cells (Testa et al., Biomark Res., 10; 2(1):4. (2014), Jordan et al., Leukemia, 14(10):1777-84 (2000)). However, CD123 is overexpressed in acute myeloid leukemia (AML) (Testa 2014). An immunoconjugate that targets CD123 called IMGN632 or pivekimab sunirine (pvek) has been produced. This immunoconjugate contains a high-affinity anti-CD123 antibody, a cleavable linker, and an indolinobenzodiazepine pseudodimer (IGN) payload. The IGN payload alkylates DNA and causes single strand breaks without crosslinking. IGNs are designed to have high potency against tumor cells, while demonstrating less toxicity to normal marrow progenitors than other DNA-targeting payloads. Pivekimab sunirine exhibited potent antitumor activity in AML xenografts, while demonstrating less toxicity in normal marrow progenitors versus other DNA-targeting payloads. Given the inability of currently available therapeutics to adequately treat AML, there is a need for more effective interventions.

### BRIEF SUMMARY OF THE INVENTION

Provided herein is a method of treating an unfit newly diagnosed acute myeloid leukemia (ND AML) in a subject comprising administering to the subject a combination therapy comprising (i) pivekimab sunirine, (ii) a BCL-2 inhibitor, and (iii) a hypomethylating agent.

In some aspects provided herein, the administration achieves a minimal residual disease (MRD)-negative status in 2 months or less.

In some aspects provided herein, the ND AML is a FLT3 TKD, IDH1^{mut}, NMP1^{mut}, and/or K/NAS^{mut} AML.

Also provided herein is a method for treating a FLT3 TKD, IDH1^{mut}, NMP1^{mut}, and/or K/NAS^{mut} acute myeloid leukemia (AML) in a subject comprising administering to the subject (i) pivekimab sunirine, (ii) a BCL-2 inhibitor, and (iii) a hypomethylating agent.

In some aspects provided herein, the administration of the combination therapy achieves a complete response with a duration of at least 3 months.

Also provided herein is a method for treating an acute myeloid leukemia (AML) in a subject comprising administering to the subject a combination therapy comprising (i) pivekimab sunirine, (ii) a BCL-2 inhibitor, and (iii) a hypomethylating agent, wherein the administration achieves a CR, CRh, CRp, or CRi with a duration of at least 3 months.

In some aspects provided herein, the duration of the CR, CRh, CRp, or CRi is at least 4 months. In some aspects provided herein, the duration of the CR, CRh, CRp, or CRi is at least 5 months. In some aspects provided herein, the duration of the CR, CRh, CRp, or CRi is at least 6 months. In some aspects provided herein, the duration of the CR, CRh, CRp, or CRi is at least 10 months. In some aspects provided herein, the duration of the CR, CRh, CRp, or CRi is at least 12 months.

In some aspects provided herein, the method further comprises administering a hematopoietic stem cell transplant (HSCT) to the subject. In some aspects provided herein, the HSCT is allogeneic. In some aspects provided herein, the HSCT is autologous. In some aspects provided herein, the HSCT is administered about 4 to about 8 weeks after an administration of pivekimab sunirine.

Also provided herein is a method for treating an acute myeloid leukemia (AML) in a subject comprising administering to the subject pivekimab sunirine, wherein the administration achieves a complete response (CR), CR (clinical) with partial hematological recovery (CRh) or CR (clinical) with incomplete recovery (CRi), in about 3 months or less.

In some aspects provided herein, the CR, CRh, or CRi had a duration of a least 2 months.

Also provided herein is a method for treating an acute myeloid leukemia (AML) in a subject comprising administering to the subject pivekimab sunirine, wherein the administration achieves a complete response (CR), CR (clinical) with partial hematological recovery (CRh) or CR (clinical) with incomplete recovery (CRi) with a duration of at least 2 months.

In some aspects provided herein, the administration of pivekimab sunirine achieves a CR, CRh, or CRi in about 2.5 months or less.

In some aspects provided herein, the method further comprises administering a hematopoietic stem cell transplant (HSCT) to the subject.

Also provided herein is a method of preparing a subject with acute myeloid leukemia (AML) for a hematopoietic stem cell transplant (HSCT), the method comprising administering to the subject pivekimab sunirine.

Also provided herein is a method for treating an acute myeloid leukemia (AML) in a subject comprising (i) administering to the subject pivekimab sunirine and (ii) subsequently administering a hematopoietic stem cell transplant (HSCT) to the subject.

Also provided herein is a method for treating an acute myeloid leukemia (AML) in a subject comprising administering a hematopoietic stem cell transplant (HSCT) to the subject, wherein the subject has previously been treated with pivekimab sunirine.

In some aspects provided herein, the HSCT is allogeneic. In some aspects provided herein, the HSCT is autologous. In some aspects provided herein, the HSCT is administered about 4 to about 8 weeks after the administration of pivekimab sunirine.

In some aspects provided herein, the administration of pivekimab sunirine achieves a minimal residual disease (MRD)-negative status.

In some aspects provided herein, the AML is a FLT3 TKD, IDH1^{mut}, NMP1^{mut}, and/or K/NAS^{mut} AML.

In some aspects provided herein, the pivekimab sunirine is administered once every three weeks (Q3W).

In some aspects provided herein, the method further comprises administering a BCL-2 inhibitor, a hypomethylating agent, or a combination thereof.

In some aspects provided herein, the administration of pivekimab sunirine or the combination therapy results in survival for at least 3 months. In some aspects provided herein, the administration of pivekimab sunirine or the combination therapy results in survival for at least 6 months. In some aspects provided herein, the administration of pivekimab sunirine or the combination therapy achieves undetectable disease.

In some aspects provided herein, the administration results in an pivekimab sunirine Cmax of about 400 to about 600 ng/mL after a single administration of the pivekimab sunirine and/or a pivekimab sunirine Cmax of about 550 to 700 ng/mL after three administrations of the pivekimab sunirine.

In some aspects provided herein, the administration results in an pivekimab sunirine AUC_{INF} of about 4,500 to about 5,000 h*ng/mL after a single administration of the pivekimab sunirine and a pivekimab sunirine AUC_{INF} of about 4,750 to about 5,250 h*ng/mL after three administrations of the pivekimab sunirine.

In some aspects provided herein, the administration of pivekimab sunirine or the combination therapy results in no more than 30% CD123 receptor availability 4 hours after administration of the pivekimab sunirine.

In some aspects provided herein, the AML is a TP53^{WT} AML. In some aspects provided herein, the AML is a K/NRAS^{WT} AML with no FLT3-ITD. In some aspects provided herein, the AML is a FLT3-ITD or K/NRAS^{mut} AML.

In some aspects provided herein, the AML is a TP53^{mut} AML. In some aspects provided herein, the AML is a FLT3 ITD or TKD, IDH1^{mut}, IDH2^{mut}, NPM1^{mut}, and/or K/NRAS^{mut} AML.

In some aspects provided herein, the subject has a baseline mutation in RUNX1, ASXL1, or TP53.

In some aspects provided herein, the AML has a favorable ELN 2017 risk. In some aspects provided herein, the AML has an intermediate ELN 2017 risk. In some aspects provided herein, the AML has an adverse ELN 2017 risk.

In some aspects provided herein, the subject was previously treated with an anti-CD123 therapy, optionally wherein the anti-CD123 therapy was tagraxofusp (SL-401). In some aspects provided herein, the subject has not received tagraxofusp (SL-401) prior to the administration of pivekimab sunirine or the combination therapy.

In some aspects provided herein, the AML expresses multidrug resistance 1 (MDR1). In some aspects provided herein, the AML expresses P-glycoprotein (P-gp).

In some aspects provided herein, the subject has an absolute neutrophil count of greater than 500/μL (microliter).

In some aspects provided herein, the AML is unfit AML. In some aspects provided herein, the AML is fit AML.

In some aspects provided herein, the cancer has previously been treated with venetoclax. In some aspects provided herein, the cancer has not previously been treated with venetoclax.

In some aspects provided herein, the cancer has previously been treated with a hypomethylating agent. In some aspects provided herein, the cancer has not previously been treated with a hypomethylating agent.

In some aspects provided herein, the subject received a stem cell transplant prior to the administration of pivekimab sunirine or the combination therapy. In some aspects provided herein, the stem cell transplant was received at least 120 days prior to the administration of pivekimab sunirine or the combination therapy. In some aspects provided herein, the previous stem cell transplant was allogeneic. In some aspects provided herein, the previous stem cell transplant was autologous.

In some aspects provided herein, the AML is a relapsed and/or refractory AML. In some aspects provided herein, the AML is a relapsed AML. In some aspects provided herein, the AML is a refractory AML.

In some aspects provided herein, the subject received at least one prior line of therapy, at least two prior lines of therapy, or at least three prior lines of therapy. In some aspects provided herein, the administration of pivekimab sunirine or the combination therapy is a frontline therapy.

In some aspects provided herein, the AML is de novo AML.

In some aspects provided herein, the subject has a prior or concomitant hematologic malignancy (PCHM). In some aspects provided herein, the PCHM does not require immediate therapy. In some aspects provided herein, the PCHM is in remission and the subject has completed all therapies for the PCHM at least 6 months prior to the administration of pivekimab sunirine or the combination therapy.

In some aspects provided herein, the subject has not received immunosuppressive treatment for at least 14 days prior to the administration of pivekimab sunirine or the combination therapy. In some aspects provided herein, the subject has not received a systemic anti-cancer therapy for at least 14 days prior to the administration of pivekimab sunirine or the combination therapy. In some aspects provided herein, the subject has received a local therapy at least 14 days prior to the administration of pivekimab sunirine or the combination therapy. In some aspects provided herein, the therapy was radiotherapy.

In some aspects provided herein, the method further comprises administration of ursodeoxycholic acid.

In some aspects provided herein, the subject has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

In some aspects provided herein, the pivekimab sunirine is administered intravenously. In some aspects provided herein, the pivekimab sunirine is administered in an infusion having a duration of no more than 30 minutes.

In some aspects provided herein, the pivekimab sunirine is administered in an infusion having a duration of about 15 to 30 minutes. In aspects, the pivekimab sunirine is administered at an infusion rate of from about 0.8 m/min to about 1.7 mL/min. In some aspects, the pivekimab sunirine is administered at an infusion rate of about 0.8 m/min (about 50 mL/hour or about 0.165 mg/min) for the first 30 minutes. In aspects, the infusion rate may be increased to about 1.7 mL/min (100 mL/hour or 0.33 mg/min). In aspects, the infusion rate may be increased to about 1.7 m/min (100 mL/hour or 0.33 mg/min), if well tolerated whereby no infusion-related reactions are observed in the patient. In aspects, the pivekimab sunirine is not administered by IV push.

In some aspects provided herein, the pivekimab sunirine is administered at a dose of about 0.045 mg/kg. In some aspects provided herein, the immunoconjugate is administered once every 4 weeks (Q4W).

In some aspects provided herein, the BCL-2 inhibitor is administered daily for at least 14 days of a 28-day cycle. In some aspects provided herein, the BCL-2 inhibitor is administered daily for up to 28 days of a 28-day cycle. In some aspects provided herein, the BCL-2 inhibitor is administered at a dose of up to about 400 mg. In some aspects provided herein, the BCL-2 inhibitor is administered orally. In some aspects provided herein, the BCL-2 inhibitor is venetoclax.

In some aspects provided herein, the hypomethylating agent is administered daily for days 1-7 of a 28-day cycle. In some aspects provided herein, the hypomethylating agent is administered at a dose of about 75 mg/m². In some aspects provided herein, the hypomethylating agent is administered subcutaneously or intravenously. In some aspects provided herein, the hypomethylating agent is azacitidine. In some aspects provided herein, the hypomethylating agent is decitabine.

In some aspects provided herein, the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for at least 14 days.

In some aspects provided herein, the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for up to 28 days.

In some aspects provided herein, the administration of pivekimab sunirine or the combination therapy is for one cycle. In some aspects provided herein, the administration of pivekimab sunirine or the combination therapy is for more than one cycle, optionally wherein the administration is for at least 2 cycles, at least 3 cycles, at least 4 cycles, at least 5 cycles, at least 6 cycles, at least 7 cycles, at least 8 cycles, at least 9 cycles, or at least 10 cycles or wherein the administration is for about 2-4 cycles, about 2-6 cycles, about 2-8 cycles, about 2-10 cycles, or about 2-12 cycles.

In some aspects provided herein, the method further comprises administering a reduced dose of the pivekimab sunirine after a dose-limiting toxicity has occurred in the subject and has been reduced to baseline or ≤Grade 2.

In some aspects provided herein, the AML is CD123-positive. In some aspects provided herein, CD123 has been detected in a sample obtained from the AML prior to the administration of pivekimab sunirine or the combination therapy, optionally wherein the CD123 was detected using flow cytometry or immunohistochemistry. In some aspects provided herein, the method further comprises detecting CD123 in a sample obtained from the AML prior to the administration of pivekimab sunirine or the combination therapy. In some aspects provided herein, at least 80% of cells in the AML express CD123. In some aspects provided herein, CD123 has been detected in at least 80% of cells in a sample obtained from the AML prior to the administration of pivekimab sunirine or the combination therapy. In some aspects provided herein, the method further comprises detecting CD123 in at least 80% of cells in a sample obtained from the AML prior to the administration of pivekimab sunirine or the combination therapy.

In some aspects provided herein, the subject is not selected based on level of CD123 expression.

In some aspects provided herein, the subject has been pretreated with a premedication prior to administration of the pivekimab sunirine, optionally wherein the premedication is a corticosteroid. In some embodiments, premedications administered prior to the administration of the pivekimab sunirine may be one or more of diphenhydramine, acetaminophen, paracetamol, dexamethasone, or a combination thereof.

In some aspects provided herein, the method further comprises pre-treating the subject with a premedication or corticosteroid prior to administration of the pivekimab sunirine, optionally wherein the premedication or corticosteroid is diphenhydramine, acetaminophen, paracetamol, dexamethasone, or a combination thereof.

Also provided herein is the use of pivekimab sunirine in any method provided herein or in the preparation for a medicament for use in any method provided herein.

### BRIEF DESCRIPTION OF THE FIGURES

FIG. 1 shows the best decrease in bone marrow blasts (%) in all cohort 1 and cohort 2 R/R AML patients treated with pivekimab sunirine monotherapy. Eight participants are not represented due to missing bone marrow (BM) data; 11 participants had >100% increase in BM blasts.

FIG. 2 shows anti-leukemia responses in efficacy-evaluable participants with R/R AML across all doses of pivekimab sunirine monotherapy. CR, complete remission; CRh, CR with partial hematologic recovery; CRi, CR with incomplete recovery; MLS, morphologic leukemia-free state; PR, partial remission.

FIG. 3 shows CD123 expression versus best overall response in R/R AML patients following pivekimab sunirine monotherapy dosing in cycle 1. ABC, antibody binding capacity or antibody bound per cell; PD, progressive disease; SD, stable disease.

FIG. 4 shows non-hematological TEAEs (>20%) by Cohort in patients with newly diagnosed AML treated with pivekimab sunirine, azacitidine, and venetoclax.

FIG. 5A shows the count recovery curve of overall time to absolute neutrophil count (ANC) recovery (≥500 μL) in newly diagnosed AML patients treated with pivekimab sunirine, azacitidine, and venetoclax in efficacy evaluable patients who demonstrated a CR, CRi, CRp, or CRh in blast clearance cycles.

FIG. 5B shows the count recovery curve of overall time to platelet recovery (>50,000 μL) in newly diagnosed AML patients treated with pivekimab sunirine, azacitidine, and venetoclax in efficacy evaluable patients who demonstrated a CR, CRi, CRp, or CRh in blast clearance cycles.

FIG. 6 shows the best change in blast numbers from baseline in the overall population of newly diagnosed AML patients treated with pivekimab sunirine, azacitidine, and venetoclax.

FIG. 7 shows the duration of overall response in months since first dose in newly diagnosed AML patients treated with pivekimab sunirine, azacitidine, and venetoclax.

### DETAILED DESCRIPTION OF THE INVENTION

### I. Definitions

To facilitate an understanding of the present invention, a number of terms and phrases are defined below.

The terms “IL-3Rα,” “Interleukine-3 Receptor alpha,” and “CD123,” as used interchangeably herein, refer to mammalian CD123 polypeptides, including, but not limited to, native CD123 polypeptides and isoforms of CD123 polypeptides, unless otherwise indicated. The terms encompass “full-length,” unprocessed CD123 polypeptides as well as any form of CD123 polypeptide that results from processing within the cell. The term also encompasses naturally occurring variants of CD123, e.g., those encoded by splice variants and allelic variants. The CD123 polypeptides described herein can be isolated from a variety of sources, such as from human tissue types or from another source, or prepared by recombinant or synthetic methods. Where specifically indicated, “CD123” can be used to refer to a nucleic acid that encodes a CD123 polypeptide. Human CD123 sequences are known and include, for example, those sequences associated with NCBI reference numbers NP_002174 & NM_002183 (protein and nucleic acid sequences for human CD123 variant 1), and NP_001254642 & NM_001267713 (protein and nucleic acid sequences for human CD123 variant 2).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “anti-CD123 antibody” or “an antibody that binds to CD123” refers to an antibody that is capable of binding CD123 with sufficient affinity such that the antibody is useful as a diagnostic and/or therapeutic agent in targeting CD123 (e.g., the antibody in pivekimab sunirine). The extent of binding of an anti-CD123 antibody to an unrelated, non-CD123 protein can be less than about 10% of the binding of the antibody to CD123 measured, e.g., by a radioimmunoassay (RIA).

The term “antibody fragment” refers to a portion of an intact antibody with a sufficient positive charge to bind to a cation exchange resin. An “antigen-binding fragment” refers to a portion of an intact antibody that binds to an antigen and has a sufficient positive charge to bind to a cation exchange resin. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “cysteine engineered” antibody or antigen-binding fragment thereof includes an antibody or antigen-binding fragment thereof with at least one cysteine (“Cys”) that is not normally present at a given residue of the antibody or antigen-binding fragment thereof light chain or heavy chain. Such Cys, which may also be referred to as “engineered Cys,” can be engineered using any conventional molecular biology or recombinant technology (e.g., by replacing the coding sequence for a non-Cys residue at the target residue with a coding sequence for Cys). For example, if the original residue is Ser with a coding sequence of 5′-UCU-3′, the coding sequence can be mutated (e.g., by site-directed mutagenesis) to 5′-UGU-3′, which encodes Cys. In certain aspects, the Cys engineered antibody or antigen-binding fragment thereof has an engineered Cys in the heavy chain. In certain aspects, the engineered Cys is in or near the CH3 domain of the heavy chain. In certain aspects, the engineered Cys is at residue 442 of the heavy chain (EU/OU numbering; EU index, Kabat et al, Sequences of Proteins of Immunological Interest, 5th Ed., NIH publication No. 91-3242, 1991, the entire contents of which are incorporated herein by reference). In certain aspects, the Fc region comprises a cysteine at one or more of positions 239, 282, 289, 297, 312, 324, 330, 335, 337, 339, 356, 359, 361, 383, 384, 398, 400, 440, 422, and 442, as numbered by the EU index. In certain aspects, any one or more of the following residues may be substituted with cysteine: V205 (Kabat numbering) of the light chain; A118 (EU numbering) of the heavy chain; and S400 (EU numbering) of the heavy chain Fc region. In certain aspects, the variable light chain domain, e.g., of an scFv, has a cysteine at Kabat position 100. In certain aspects, the variable heavy chain domain, e.g., of an scFv, has a cysteine at Kabat position 44. Cysteine engineered antibodies may be generated as described, e.g., in U.S. Pat. Nos. 7,521,541, 7,855,275, U.S. Published Application No. 2011/0033378 and WO 2011/005481.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative aspects are described in the following.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain aspects, the polypeptides can occur as single chains or associated chains.

The term “immunoconjugate” or “conjugate” as used herein refers to a compound or a derivative thereof that is linked to a cell binding agent (i.e., an anti-CD123 antibody or fragment thereof) and is defined by a generic formula: C-A, wherein C=cytotoxin (e.g., such as an indolino-benzodiazepine (IGN) DNA-alkylator (e.g., DGN549-C)) and A=antibody or antigen-binding fragment thereof, e.g., an anti-CD123 antibody or antibody fragment. An immunoconjugate can optionally contain a linker and be defined by the generic formula C-L-A, wherein C=cytotoxin, L=linker, and A=antibody or antigen-binding fragment thereof, e.g., an anti-CD123 antibody or antibody fragment. Immunoconjugates can also be defined by the generic formula in reverse order: C-A or A-L-C. Immunoconjugates can also contain multiple cytotoxins (C) per antibody or antigen-binding fragment thereof (A) or multiple cytotoxins (C) and linkers (L) per antibody or antigen-binding fragment thereof (A).

A “linker” is any chemical moiety that is capable of linking a compound, usually a drug (such as IGN DNA-alkylators), to a cell-binding agent (such as an anti-CD123 antibody or a fragment thereof) in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and known in the art. In some aspects disclosed herein, the linker is a peptide linker.

The term “BCL-2 inhibitor” refers to an agent that is capable of inhibiting an activity of B-cell leukemia/lymphoma-2 (“BCL-2”). For example, a BCL-2 inhibitor can bind to BCL-2 and reduce the interaction of BCL-2 with pro-apoptotic proteins (e.g., BH3-only proteins). Venetoclax is an exemplary BCL-2 inhibitor.

The term “hypomethylating agent” or “HMA” refers to agents that inhibits DNA methylation. For example, an HMA can act by inhibiting a DNA methyltransferase. Azacitidine and decitabine are exemplary HMAs.

The terms “cancer” and “cancerous” refer to or describe the physiological condition in mammals in which a population of cells are characterized by unregulated cell growth. Examples of cancer include acute myeloid leukemia (AML). The cancer can be a cancer that expresses CD123 (“CD123-expressing cancer”).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

A “refractory” cancer is one that progresses even though an anti-cancer treatment, such as a chemotherapy, is administered to the cancer patient. The cancer may be resistant at the beginning of treatment or it may become resistant during treatment.

A “primary refractory” cancer is one that does not achieve complete remission (CR) or complete remission with incomplete recovery (CRi) after a patient has received 2 cycles of intense chemotherapy.

A “relapsed” cancer is one in which the cancer or the signs and symptoms of a cancer returns after a period of improvement.

As it relates to cancer and cancer treatment, “remission” and “response” means a decrease, improvement or disappearance of one or more signs or symptoms of cancer.

Depending on context, the term “CR” means “Complete Remission” or “Complete Response”.

Depending on context, the term “PR” means “Partial Remission” or “Partial Response”.

The term “fit AML” as used herein refers to a subject having AML who is eligible for intensive therapy. The measures for determining a subject with fit AML include, e.g., physical performance (as determined by e.g., the Eastern Cooperative Oncology Group performance status (ECOG PS), the Karnofsky performance status (KPS), and the short physical performance battery (SPPB)), comorbid conditions (as determined by the Charlson comorbidity index (CCI) or the hematopoietic cell transplantation-specific comorbidity index (HCT-CI)), cognitive function, and prognostic models (including but not limited to, cytogenetic group, age, white blood cell count, LDH, type of AML). In some cases, a fit AML subject is a subject at the age of 60 or under the age of 60.

The term “unfit AML” as used herein refers to a subject having AML who is ineligible for intensive therapy. The measures for determining a subject with unfit AML include, e.g., physical performance (as determined by e.g., the Eastern Cooperative Oncology Group performance status (ECOG PS), the Karnofsky performance status (KPS), and the short physical performance battery (SPPB)), comorbid conditions (as determined by the Charlson comorbidity index (CCI) or the hematopoietic cell transplantation-specific comorbidity index (HCT-CI)), cognitive function, and prognostic models (including but not limited to, cytogenetic group, age, white blood cell count, LDH, type of AML). In some cases, an unfit AML subject is a subject over the age of 60. The term “fit AML” as used herein refers to a subject having AML who is eligible for cancer treatment.

The term “increased expression” or “overexpression” CD123 in a particular tumor, tissue, or cell sample refers to CD123 that is present at a level higher than that which is present in a healthy or non-diseased (native, wild type) tissue or cells of the same type or origin.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder.

The term “therapeutically effective amount” refers to an amount of an antibody, immunoconjugate, or other drug effective to “treat” a disease or disorder in a subject or mammal. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; relieve to some extent one or more of the symptoms associated with the cancer; and/or result in a favorable response such as complete remission (CR), complete remission with incomplete recovery (CRi); complete remission with partial hematologic recovery (CRh); CR without minimal residual disease (CRMRD−); complete remission clinical (CRc); morphologic leukemia-free state (MLFS); partial remission (PR); duration of response (DOR); and decrease in progressive disease (PD).

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). A favorable response can be assessed, for example, by complete remission (CR), complete remission with partial hematologic recovery (CRh), complete remission/response with incomplete recovery (CRi); CR without minimal residual disease (CRMRD−); morphologic leukemia-free state (MLFS); partial response (PR); stable disease (SD), a decrease in progressive disease (PD), or any combination thereof. The assessment criteria for evaluating the treatment of AML are provided in Table 6 in Example 1 below.

The term “clinical benefit” or “(clinical)” refer to an achievement by a patient of complete response or partial response for a time equal to or longer than 4 weeks.

The terms “line of treatment” or “line of therapy” refer to a therapeutic regimen that can include but is not limited to surgery, radiation therapy, chemotherapy, differentiating therapy, biotherapy, immune therapy, induction therapy, consolidation therapy, transplant, maintenance therapy, or the administration of one or more anti-cancer agents (e.g., a cytotoxic agent and/or an anti-proliferative compound).

The terms “first-line treatment,” “first-line therapy,” and “frontline therapy” refer to the preferred and standard initial treatment for a particular condition, e.g., induction therapy, consolidation therapy, transplant, maintenance therapy. As used herein, patients who receive “frontline therapy” are patients who have not received prior systemic therapy. However such a patient may have previously received a local therapy such as radiotherapy, surgical excision and/or photodynamic therapy. These treatments differ from “second-line” therapies, which are tried when a first-line therapy does not work adequately. “Third-line” therapies are tried when a first-line therapy and a second-line therapy do not work adequately. “Salvage therapy” or “salvage regimen” is a treatment that is tried when the cancer has not responded to other treatments.

For example, a CD123 immunoconjugate (e.g. pivekimab sunirine) or the combination of a CD123 immunoconjugate with a BCL-2 inhibitor, and/or an HMA provided herein can be given as a first-line therapy, a second-line therapy, or a third-line therapy. A CD123 immunoconjugate (e.g. pivekimab sunirine) or the combination of a CD123 immunoconjugate (e.g. pivekimab sunirine) with a BCL-2 inhibitor, and/or an HMA provided herein can be given as a line of therapy in patients having received at least 1, at least 2, or at least 3 lines of therapy (e.g., front-line therapy and one salvage therapy) prior to treatment with the combination of a CD123 immunoconjugate (e.g. pivekimab sunirine) with a BCL-2 inhibitor, and/or an HMA provided herein. In some aspects, a CD123 immunoconjugate (e.g. pivekimab sunirine) or the combination of a CD123 immunoconjugate with a BCL-2 inhibitor, and/or an HMA provided herein can be given as a line of therapy in patients having received no more than 1, no more than 2, no more than 3, no more than 4, no more than 5, or no more than 6 lines of therapy.

The term “maintenance therapy” refers to therapy that is given to help keep cancer from coming back after it has disappeared following the initial therapy.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The term “premedication” refers to one or more additional agent(s) administered to a subject for a purpose that is not primarily or solely treating the primary disease (e.g., BPDCN) in the subject. For example, premedication may refer to one or more additional agent(s) used to prevent or ameliorate injection site side effects, infusion-related reactions, fever, and/or any other side effect associated with an anti-CD123 antibody. In embodiments, a premedication is administered using a premedication regimen.

The term “controlled rate of infusion” means administering a therapeutic agent in a manner that minimizes the variability of the average amount of the therapeutic agent, (e.g., pivekimab sunirine) that is administered to the patient over a period of time to, e.g., ensure a constant delivery of the therapeutic agent over a prolonged time. “Controlled rate of infusion” does not mean administration via IV push.

The term “subject” or “patient” refers to a human who is to be the recipient of a particular treatment.

The term “combination therapy” or “combination treatment” refers to administration of one or more therapeutic agents, in any order, in amounts that are effective for the cancer treatment intended. The administration of one or more therapeutic agents may be, e.g., simultaneous, concurrent, consecutive, or during a cycle of treatment.

Administration “in combination with” one or more further therapeutic agents includes simultaneous (concurrent) or consecutive administration in any order.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially, e.g., by different injections in separate syringes.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). It is understood that wherever aspects are described herein with the language “about” a numeric value or range, otherwise analogous aspects referring to the specific numeric value or range (without “about”) are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### II. Anti-CD123 Immunoconjugates

Described herein are methods of administering immunoconjugates that specifically bind CD123 (e.g., pivekimab sunirine) alone or in combination with other agents. Immunoconjugates that specifically bind to CD123 are referred to herein as “CD123-immunoconjugates” or “anti-CD123 immunoconjugates.” Such immunoconjugates comprise an anti-CD123 antibody or antigen-binding fragment thereof and a drug (e.g., a cytotoxic agent). The drug (e.g., a cytotoxic agent) can be attached to the anti-CD123 antibody or antigen-binding fragment thereof by a linker.

In some aspects, the anti-CD123 immunoconjugate is pivekimab sunirine.

In some aspects, the anti-CD123 antibodies or antigen-binding fragments thereof are humanized antibodies or antigen-binding fragments thereof. In some aspects, the humanized antibody or fragment is a resurfaced antibody or antigen-binding fragment thereof. In other aspects, the antibodies or antigen-binding fragments thereof is a fully human antibody or antigen-binding fragment thereof.

In certain aspects, the immunoconjugate is represented by the following formula:

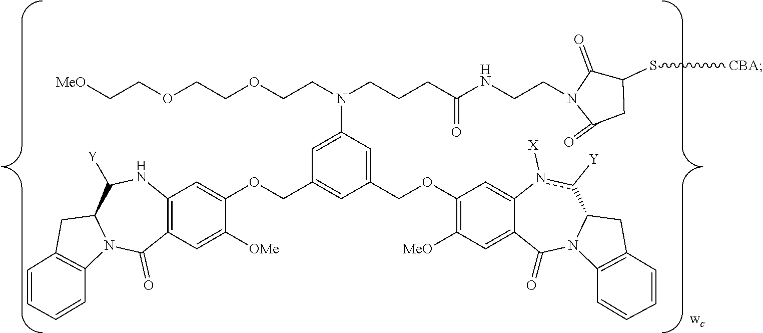

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO3M.

By way of example, an anti-CD123 antibody or antigen-binding fragment thereof can be in an immunoconjugate used in the present methods. Anti-CD123 antibodies or antigen-binding fragments thereof have been described (see e.g., U.S. Pat. No. 10,077,313 B2, the contents of which are herein incorporated by reference in their entirety). The anti-CD123 antibody or antigen-binding fragment thereof can be the huCD123-6Gv4.7 (“G4723A”) antibody (see WO 2017/004025, WO 2017/004026, and PCT/US2018/052212, the contents of each of which are herein incorporated by reference in their entireties) or can contains sequences of the G4723A antibody, e.g., as shown below in Tables 1-3. For example, an anti-CD123 antibody or antigen-binding fragment thereof for use in the methods provided herein can comprise variable heavy chain CDR-1, CDR-2, and CDR-3 comprising the sequences of SEQ ID NOs: 5, 6, and 7, respectively and/or variable light chain CDR-1, CDR-2, and CDR-3 comprising the sequences of SEQ ID NOs: 8, 9, and 10, respectively. An anti-CD123 antibody or antigen-binding fragment thereof for use in the methods provided herein can comprise a variable heavy chain domain comprising the sequence set forth in SEQ ID NO:1. An anti-CD123 antibody or antigen-binding fragment thereof for use in the methods provided herein can comprise a variable light chain domain comprising the sequence set forth in SEQ ID NO:2. An anti-CD123 antibody or antigen-binding fragment thereof for use in the methods provided herein can comprise a variable heavy chain domain comprising the sequence set forth in SEQ ID NO:1 and a variable light chain domain comprising the sequence set forth in SEQ ID NO:2. An anti-CD123 antibody or antigen-binding fragment thereof for use in the methods provided herein can comprise a heavy chain comprising the sequence set forth in SEQ ID NO:3. An anti-CD123 antibody or antigen-binding fragment thereof for use in the methods provided herein can comprise a light chain comprising the sequence set forth in SEQ ID NO:4. An anti-CD123 antibody or antigen-binding fragment thereof for use in the methods provided herein can comprise a heavy chain comprising the sequence set forth in SEQ ID NO:3 and a light chain comprising the sequence set forth in SEQ ID NO:4.

TABLE 1
huCD123-6Gv4.7 Heavy and Light Chain
Variable Regions

| Name | Sequence |
|---|---|
| huCD123-6Gv7 | QVQLVQSGAEVKKPGASVKVSCKASGYIFTSSIMHW |
| Heavy Chain | VRQAPGQGLEWIGYIKPYNDGTKYNEKFKGRATLTS |
| Variable | DRSTSTAYMELSSLRSEDTAVYYCAREGGNDYYDTM |
| Region | DYWGQGTLVTVSS (SEQ ID NO: 1) |
| huCD123-6Gv4 | DIQMTQSPSSLSASVGDRVTITCRASQDINSYLSWF |
| Light Chain | QQKPGKAPKTLIYRVNRLVDGVPSRFSGSGSGNDYT |
| Variable | LTISSLQPEDFATYYCLQYDAFPYTFGQGTKVEIKR |
| Region | (SEQ ID NO: 2) |

TABLE 2
huCD123-6Gv4.7-C442 Full Length Heavy and
Light Chain

| Name | Sequence |
|---|---|
| huCD123- | QVQLVQSGAEVKKPGASVKVSCKASGYIFTSSIMHWVR |
| 6Gv7-C442 | QAPGQGLEWIGYIKPYNDGTKYNEKFKGRATLTSDRST |
| Full Length | STAYMELSSLRSEDTAVYYCAREGGNDYYDTMDYWGQG |
| Heavy Chain | TLVTVSSASTKGPSVFPLAPSSKSTSGGTAALGCLVKD |
| | YFPEPVTVSWNSGALTSGVHTFPAVLQSSGLYSLSSVV |
| | TVPSSSLGTQTYICNVNHKPSNTKVDKKVEPKSCDKTH |
| | TCPPCPAPELLGGPSVFLFPPKPKDTLMISRTPEVTCV |
| | VVDVSHEDPEVKFNWYVDGVEVHNAKTKPREEQYNSTY |
| | RVVSVLTVLHQDWLNGKEYKCKVSNKALPAPIEKTISK |
| | AKGQPREPQVYTLPPSRDELTKNQVSLTCLVKGFYPSD |
| | IAVEWESNGQPENNYKTTPPVLDSDGSFFLYSKLTVDK |
| | SRWQQGNVFSCSVMHEALHNHYTQKSLCLSPG (SEQ |
| | ID NO: 3) |
| huCD123- | DIQMTQSPSSLSASVGDRVTITCRASQDINSYLSWFQQ |
| 6Gv4 | KPGKAPKTLIYRVNRLVDGVPSRFSGSGSGNDYTLTIS |
| Full Length | SLQPEDFATYYCLQYDAFPYTFGQGTKVEIKRTVAAPS |
| Light Chain | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC (SEQ ID NO: |
| | 4) |

TABLE 3
huCD123-6Gv4.7 Variable Heavy and Light Chain
Complementary Determining Regions

| Name | Sequence |
|---|---|
| huCD123-6Gv7 Variable | SSIMH |
| Heavy Chain CDR1 | (SEQ ID NO: 5) |
| huCD123-6Gv7 Variable | YIKPYNDGTKYNEKFKG |
| Heavy Chain CDR2 | (SEQ ID NO: 6) |
| huCD123-6Gv7 Variable | EGGNDYYDTMDY |
| Heavy Chain CDR3 | (SEQ ID NO: 7) |
| huCD123-6Gv4 Variable | RASQDINSYLS |
| Light Chain CDR1 | (SEQ ID NO: 8) |
| huCD123-6Gv4 Variable | RVNRLVD |
| Light Chain CDR2 | (SEQ ID NO: 9) |
| huCD123-6Gv4 Variable | LQYDAFPYT |
| Light Chain CDR3 | (SEQ ID NO: 10) |

An anti-CD123 antibody or antigen-binding fragment thereof for use in the methods provided herein can bind to an epitope within amino acids 205 to 346 of human CD123.

An anti-CD123 antibody or antigen-binding fragment thereof for use in methods provided herein can be recombinantly produced. For example, an anti-CD123 antibody or antigen-binding fragment thereof for use in the methods provided herein can be produced in a mammalian cell line, e.g., a CHO cell.

An anti-CD123 antibody or antigen-binding fragment thereof for use in the methods provided herein can be a cysteine-engineered antibody or fragment. Cysteine-engineered antibodies can be covalently conjugated to cytotoxic agents of interest to generate immunoconjugates.

As used herein, the expression “linked to a cell-binding agent” or “linked to an anti-CD123 antibody or fragment” refers to a conjugate molecule comprising at least one cytotoxic agent bound to a cell-binding agent, e.g., anti-CD123 antibody or fragment, via a suitable linking group, or a precursor thereof. Linkers include, for example, peptide linkers.

An immunoconjugate can contain multiple cytotoxic agents bound to an antibody or antigen-binding fragment thereof. As provided herein, in certain instances, about 1 to about 3 drug molecules e.g., cytotoxic agents, are linked to an anti-CD123 antibody or antigen-binding fragment thereof. In one aspect, an immunoconjugate comprises 1, 2, or 3, cytotoxic agents per antibody or antigen-binding fragment thereof.

A composition comprising immunoconjugates can contain immunoconjugates with varying numbers of cytotoxic agents bound per antibody or antigen-binding fragment thereof. Thus, compositions comprising immunoconjugates can contain an average number of cytotoxic agents bound per antibody or antigen-binding fragment thereof. In one aspect, a pharmaceutical composition comprising anti-CD123 immunoconjugates comprises about 1 to about 3 cytotoxic agents per anti-CD123 antibody or antigen-binding fragment thereof, about 1.5 to about 2.5 cytotoxic agents per anti-CD123 antibody or antigen-binding fragment thereof, about 1.5 to about 2.1 cytotoxic agents per anti-CD123 antibody or antigen-binding fragment thereof, or about 1.5 to about 2.0 cytotoxic agents cytotoxic agents per anti-CD123 antibody or antigen-binding fragment thereof.

In certain instances, a pharmaceutical composition for use in the methods provided herein comprises anti-CD123 immunoconjugates comprising about 1 to about 3 cytotoxic agents per antibody or antigen-binding fragment thereof, for example, wherein the average number of cytotoxic agents per antibody or antigen-binding fragment thereof is from about 1 to about 3 (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0).

In certain instances, a pharmaceutical composition for use in the methods provided herein comprises anti-CD123 immunoconjugates with an average of about 1±0.2, about 1.1±0.2, about 1.2±0.2, about 1.3±0.2, about 1.4±0.2, about 1.5±0.2, about 1.6±0.2, about 1.7±0.2, about 1.8±0.2, about 1.9±0.2, about 2.0±0.2, about 2.1±0.2, 2.2±0.2, 2.3±0.2, 2.4±0.2, 2.5±0.2, or 2.6±0.2 drug molecules (e.g., cytotoxic agents) attached per antibody or antigen-binding fragment thereof. In certain aspects, a pharmaceutical composition provided herein comprises anti-CD123 immunoconjugates with an average of about 1.5 to 2.1 drug molecules (e.g., cytotoxic agents) per antibody.

The antibodies or antigen-binding fragments thereof for use in the present disclosure may be linked to cytotoxic agents, for example, through linkage with the Lys side chain amino group, the Cys side chain thiol group, or an oxidized N-terminal Ser/Thr. Cytotoxic agents include, for example, DNA alkylating agents such as indolino-benzodiazepine (IGN) DNA alkylators. In certain instances, a cytotoxic agent is an indolino-benzodiazepine pseudodimer. In certain instances, an anti-CD123 immunoconjugate for use in the present disclosure comprises DGN549-C.

### III. BCL-2 Inhibitors

Described herein are methods of administering anti-CD123 immunoconjugates such as pivekimab sunirine in combination with BCL-2 inhibitors. Overexpression of BCL-2 has been demonstrated in AML cells, where it mediates tumor cell survival and has been associated with resistance to chemotherapeutics. BCL-2 inhibitors can reverse this effect, e.g., by promoting apoptosis.

BCL-2 inhibitors include, for example, venetoclax (VENCLEXTA®; AbbVie Inc.), GX15-070 (obatoclax mesylate; GeminX Biotechnologies), AT-101 (Ascenta Therapeutics) and ABT-263 (navitoclax; Abbott) chemotherapy drugs.

Venetoclax (also known as 4-(4-{[2-(4-chlorophenyl)-4,4 dimethylcyclohex-1-en-1-yl]methyl}piperazin-1-yl)-N-({3-nitro-4-[(tetrahydro-2H-pyran-4 ylmethyl)amino]phenyl}sulfonyl)-2-(1H-pyrrolo[2,3-b]pyridin-5-yloxy)benzamide)) is a selective inhibitor of BCL-2. Venetoclax is believed to help restore the process of apoptosis by binding directly to the BCL-2 protein, displacing pro-apoptotic proteins like BIM, triggering mitochondrial outer membrane permeabilization and the activation of caspases. Venetoclax is the active ingredient in VENCLEXTA® chemotherapy medication, which is provided as a tablets for oral administration.

In some aspects, the BCL-2 inhibitor is a small molecule. In some aspects, the BCL-2 inhibitor is venetoclax.

### IV. Hypomethylating Agents

Described herein are methods of administering anti-CD123 immunoconjugates such as pivekimab sunirine in combination with hypomethylating agents, e.g., azacitidine or decitabine.

Azacitidine (also known as “4-amino-1-beta-D-ribofuranosyl-s-triazin-2(1H)-one” or “5-azacytine”) is a pyrimidine nucleoside analogue. It is thought to induce antineoplastic activity via two mechanisms: inhibition of DNA methyltransferase at low doses, causing hypomethylation of DNA, and direct cytotoxicity in abnormal hematopoietic cells in the bone marrow through its incorporation into DNA and RNA at high doses, resulting in cell death. Azacitine is the active ingredient in VIDAZA® chemotherapy medication, which is provided as a sterile form for reconstitution as a suspension for subcutaneous injection or reconstitution as a solution with further dilution for intravenous administration.

Decitabine (also known as 4-amino-1-(2-deoxy-β-D-erythro-pentofuranosyl)-1,3,5-triazin2(1H)-one) is an analog of nucleoside 2′-deoxycytidine. It is believed to exert its antineoplastic effects after phosphorylation and direct incorporation into DNA and inhibition of DNA methyltransferase, causing hypomethylation of DNA and cellular differentiation or apoptosis. Decitabine is the active ingredient in DACOGEN® chemotherapy medication, which is provided as a sterile lyophilized powder for reconstitution for intravenous administration.

Administration of an HMA in combination with an anti-CD123 immunoconjugate (e.g., pivekimab sunirine) can reduce the amount and/or frequency of HMA required to achieve the same efficacy, thereby reducing the toxicity of the therapy. Administration of an HMA in combination with an anti-CD123 immunoconjugate (e.g., pivekimab sunirine) can also increase the efficacy of the therapy.

In some aspects, the HMA is a small molecule. In some aspects, the HMA is azacitidine. In some aspects, the HMA is decitabine.

### V. Methods of Use

As provided herein, anti-CD123 immunoconjugates such as pivekimab sunirine can be used alone (as a monotherapy) or in combination with BCL-2 inhibitors and/or hypomethylating agents (HMAs) to treat AML in a subject. In some aspects, the methods comprise administering the anti-CD123 immunoconjugate to the subject. In some aspects, the method does not comprise administering a hematopoietic stem cell transplant (HSCT) to the subject to the subject.

As provided herein, anti-CD123 immunoconjugates such as pivekimab sunirine can also be used alone or in combination with BCL-2 inhibitors and/or HMAs to prepare a subject with AML for HSCT. In some aspects, provided herein are methods of treating AML in a subject by administering an anti-CD123 immunoconjugate alone or in combination with BCL-2 inhibitors and/or HMAs to the subject and then an HSCT to the subject. In some aspects, provided herein are methods of treating AML in a subject by administering a HSCT to the subject, wherein the subject was previously treated with an anti-CD123 immunoconjugate such as pivekimab sunirine. The HSCT can be allogeneic. Alternatively, the HSCT can be autologous.

The risks of infusion-related reactions may be heightened if patients are administered pivekimab sunirine by IV push or via an infusion rate that is not a controlled rate of infusion. In some aspects, the anti-CD123 immunoconjugate, e.g., pivekimab sunirine, is administered intravenously, e.g., in an infusion having a duration of no more than 30 minutes. In aspects, the pivekimab sunirine is administered via an infusion having a duration of about 15 to about 30 minutes. In aspects, the pivekimab sunirine is administered by a controlled rate of infusion. In aspects, the pivekimab sunirine is administered by a controlled rate of infusion, where the infusion rate is from about 0.8 mL/min to about 1.7 mL/min. In some aspects, the pivekimab sunirine is administered at an infusion rate of about 0.8 mL/min (about 50 mL/hour or about 0.165 mg/min) for the first 30 minutes. In aspects, the infusion rate may be increased to about 1.7 mL/min (about 100 mL/hour or about 0.33 mg/min). In aspects, the infusion rate may be increased to about 1.7 mL/min (about 100 mL/hour or about 0.33 mg/min), if well tolerated whereby no infusion-related reactions are observed in the patient. In aspects, the pivekimab sunirine is not administered by IV push. In embodiments, administering the pivekimab sunirine by a controlled rate of infusion, where the infusion rate is from about 0.8 mL/min to about 1.7 mL/min may prevent the patient from experiencing infusion-related reactions or minimize the effects of infusion-related reactions. In aspects, the pivekimab sunirine is administered via an infusion having a duration of 15 to 30 minutes. In aspects, the pivekimab sunirine is administered by a controlled rate of infusion. In aspects, the pivekimab sunirine is administered by a controlled rate of infusion, where the infusion rate is from 0.8 mL/min to 1.7 m/min. In some aspects, the pivekimab sunirine is administered at an infusion rate of 0.8 mL/min (50 mL/hour or 0.165 mg/min) for the first 30 minutes. In aspects, the infusion rate may be increased to 1.7 mL/min (100 mL/hour or 0.33 mg/min). In aspects, the infusion rate may be increased to 1.7 mL/min (100 mL/hour or 0.33 mg/min), if well tolerated whereby no infusion-related reactions are observed in the patient. In aspects, the pivekimab sunirine is not administered by IV push. In embodiments, administering the pivekimab sunirine by a controlled rate of infusion, where the infusion rate is from 0.8 m/min to 1.7 m/min may prevent the patient from experiencing infusion-related reactions or minimize the effects of infusion-related reactions.

### V(A). Cancer Selection

Cancers that can be treated by the methods provided herein include AML. In certain aspects, the AML is a CD123-expressing AML. The AML can be a newly diagnosed (ND) AML. In certain aspects, the ND AML is unfit ND AML. In certain aspects, the ND AML is fit ND AML. The AML can also be a relapsed or refractory (R/R) AML.

In certain aspects, the AML is a newly diagnosed AML. In some aspects, the AML is de novo. In some aspects, the subject has a prior or concomitant hematological malignancy (PCHM). In some aspects, the PCHM does not require immediate therapy. In some aspects, the PCHM is in remission and the subject has completed all therapies for the PCHM at least 6 months prior to the administration of the anti-CD123 immunoconjugate, e.g., pivekimab sunirine.

In certain aspects, the AML is a relapsed AML. In certain aspects, the AML is a refractory AML. In certain aspects, the AML is not secondary AML. In certain aspects, the AML is fit AML. In certain aspects, the AML is unfit AML.

In certain aspects, the AML is a FLT3 ITD or TKD, IDH1^{mut}, IDH2^{mut}, NPM1^{mut}, and/or K/NRAS^{mut} AML.

In certain aspects, the AML is a FLT3 TKD, IDH1^{mut}, NMP1^{mut}, and/or K/NAS^{mut} AML. In certain aspects, the AML is a FLT3 TKD AML. In certain aspects, the AML is a IDH1^{mut} AML. In certain aspects, the AML is a NMP1^{mut} AML. In certain aspects, the AML is a K/NAS^{mut} AML.

In certain aspects, the AML is a TP53^{WT} AML. In certain aspects, the TP53^{WT} AML is a K/NRAS^{WT} AML with no FLT3-ITD. In certain aspects, the TP53^{WT} AML is a FLT3-ITD or K/NRAS^{mut} AML.

In certain aspects, the AML is a TP53^{mut} AML.

In certain aspects, the subject has a baseline mutation in RUNX1, ASXL1, or TP53.

In certain aspects, the AML expresses multidrug resistance 1 (MDR1). In certain aspects, the AML expresses the multidrug resistance (MDR)-related P-glycoprotein (P-gp). In certain aspects, the AML overexpresses MDR1 and P-gp.

In certain aspects, the AML or the subject with the AML has an FLT3-ITD mutation. In certain aspects, the AML or the subject with the AML does not have an FLT3-ITD mutation. In certain aspects, the AML or the subject with the AML has an FLT3-TKD mutation. In certain aspects, the AML or the subject with the AML does not have an FLT3-TKD mutation.

In certain aspects, the AML has a favorable ELN 2017 risk. In certain aspects, the AML has an intermediate ELN 2017 risk. In certain aspects, the AML has an adverse ELN 2017 risk

In certain aspects, the AML is present in the subject as minimal residual disease (MRD). In certain aspects, an MRD+ patient has fit AML. In certain aspects, an MRD+ patient has unfit AML. The methods provided herein can covert MRD+ patients to MRD− patients. The methods provided herein can also increase the relapse-free survival time (e.g., the median relapse-free survival time) in MRD+ patients.

In some aspects, at least about 80% of cells of the AML are CD123+ (e.g., as determined by local flow cytometry or immunohistochemistry).

In some aspects, it has been determined prior to the administration of the anti-CD123 immunoconjugate (e.g., pivekimab sunirine) that at least 80% of cells of the AML are CD123-positive (e.g., as determined by local flow cytometry or immunohistochemistry).

In some aspects, the AML has not previously been treated. In some aspects, the AML has not been previously treated with a systemic therapy.

In some aspects, the subject has received one prior treatment regimen (e.g. one prior systemic treatment regimen) for the AML. In some aspects, the subject has received at least one prior treatment regimen (e.g., at least one prior systemic treatment regimen) for the AML. In some aspects, the subject has received two prior treatment regimens (e.g., two prior systemic treatment regimens) for the AML. In some aspects, the subject has received at least two prior treatment regimens (e.g., at least two prior systemic treatment regimens) for the AML. In some aspects, the subject has received at least three prior treatment regimens (e.g., three prior systemic treatment regimens) for the AML. In some aspects, the subject has received three prior treatment regimens (e.g., at least three prior systemic treatment regimens) for the AML. In some aspects, the subject has received one to three prior treatment regimens (e.g. at one to three prior systemic treatment regimens) for the AML. In some aspects, the subject has received no more than six prior treatment regimens (e.g., no more than six prior systemic treatment regimens) for the AML. In some aspects, the subject has received at least one prior treatment, but no more than six prior treatment regimens (e.g., at least one, but no more than six, prior systemic treatment regimens) for the AML.

In some aspects, the AML has previously been treated with venetoclax; a hypomethylating agent; cyclophosphamide, vincristine sulfate, and prednisone (CVP chemotherapy combination); and/or steroids. In some aspects, the AML has previously been treated with cyclophosphamide, vincristine sulfate, doxorubicin hydrochloride, methotrexate, cytarabine, and dexamethasone (HyperCVAD chemotherapy regimen); fludarabine, high-dose cytarabine, and G-CSF (FLAG chemotherapy combination) and/or cyclophosphamide, vincristine and doxorubicin (CHOP chemotherapy regimen).

In one aspect the AML has previously been treated with a BCL-2 inhibitor. In one aspect, the AML has not previously been treated with a BCL-2 inhibitor (i.e., the patient is “BCL-2 inhibitor naive”).

In one aspect the AML has previously been treated with venetoclax. In one aspect, the AML has not previously been treated with venetoclax (i.e., the patient is “venetoclax naive”).

In one aspect the AML has previously been treated with a hypomethylating agent. In one aspect, the AML has not previously been treated with a hypomethylating agent (i.e., the patient is “hypomethylating agent naive”).

In one aspect the AML has previously been treated with azacitidine. In one aspect, the AML has not previously been treated with azacitidine (i.e., the patient is “azacitidine naive”).

In one aspect the AML has previously been treated with decitabine. In one aspect, the AML has not previously been treated with decitabine (i.e., the patient is “decitabine naive”).

In one aspect the AML has previously been treated with tagraxofusp. In one aspect, the AML has not previously been treated with tagraxofusp (i.e., the patient is “tagraxofusp naive”).

In one aspect, the patient had previously received a stem cell transplant (e.g., an allogeneic stem cell transplant or an autologous stem cell transplant) prior to the administration of the anti-CD123 immunoconjugate. In some aspects, the stem cell transplant was received at least 120 days prior to the administration of the anti-CD123 immunoconjugate. In another aspect, the patient has not previously received a stem cell transplant prior to the administration of the anti-CD123 immunoconjugate, e.g., pivekimab sunirine.

In one aspect, the subject received a local therapy, e.g., radiotherapy, prior to the administration of the anti-CD123 immunoconjugate, e.g., pivekimab sunirine. In one aspect, the local therapy, e.g., radiotherapy, was administered at least 14 days prior to the administration of the anti-CD123 immunoconjugate, e.g., pivekimab sunirine

In one aspect, the subject has not received immunosuppressive therapy for at least 14 days and/or has not received a systemic anti-cancer therapy for at least 14 days prior to the administration prior to the administration of the anti-CD123 immunoconjugate, e.g., pivekimab sunirine.

In one aspect, the subject to receive the anti-CD123 immunoconjugate, e.g., pivekimab sunirine, has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

### V(B). Dosing

As provided herein, an anti-CD123 immunoconjugate (e.g., pivekimab sunirine) can be administered at a particular dose and/or at particular timing intervals. Administration of anti-CD123 immunoconjugates (e.g., pivekimab sunirine) can be, for example, intravenous.

In certain aspects, the anti-CD123 immunoconjugate (e.g., pivekimab sunirine) is administered, e.g., as a monotherapy, once in a three-week (21-day) cycle.

In certain aspects, one cycle of treatment is therapeutically effective. In certain aspects, two cycles of treatment are therapeutically effective. In certain aspects, three cycles of treatment are therapeutically effective. In certain aspects, one to three cycles of treatment are therapeutically effective. In certain aspects, one to four cycles of treatment are therapeutically effective. In certain aspects, one to twelve cycles of treatment

In some aspects, patients can be treated for one cycle (e.g., a 21-day cycle), e.g., wherein the anti-CD123 immunoconjugate (e.g., pivekimab sunirine) is administered once in the cycle. In some aspects, patients can be treated for at least two cycles (e.g., 21-day cycles), e.g., wherein the anti-CD123 immunoconjugate (e.g., pivekimab sunirine) is administered once per cycle. In some aspects, patients can be treated for at least three cycles (e.g., 21-day cycles), e.g., wherein the anti-CD123 immunoconjugate (e.g., pivekimab sunirine) is administered once per cycle. In some aspects, patients can be treated for at least four cycles (e.g., 21-day cycles), e.g., wherein the anti-CD123 immunoconjugate (e.g., pivekimab sunirine) is administered once per cycle. In some aspects, patients can be treated for at least five cycles (e.g., 21-day cycles), e.g., wherein the anti-CD123 immunoconjugate (e.g., pivekimab sunirine) is administered once per cycle. In some aspects, patients can be treated for at least six cycles (e.g., 21-day cycles), e.g., wherein the anti-CD123 immunoconjugate (e.g., pivekimab sunirine) is administered once per cycle. In some aspects, patients can be treated for at least seven cycles (e.g., 21-day cycles), e.g., wherein the anti-CD123 immunoconjugate (e.g., pivekimab sunirine) is administered once per cycle. In some aspects, patients can be treated for at least eight cycles (e.g., 21-day cycles), e.g., wherein the anti-CD123 immunoconjugate (e.g., pivekimab sunirine) is administered once per cycle. In some aspects, patients can be treated for at least nine cycles (e.g., 21-day cycles), e.g., wherein the anti-CD123 immunoconjugate (e.g., pivekimab sunirine) is administered once per cycle. In some aspects, patients can be treated for at least ten cycles (e.g., 21-day cycles), e.g., wherein the anti-CD123 immunoconjugate (e.g., pivekimab sunirine) is administered once per cycle. In some aspects, patients can be treated for at least eleven cycles (e.g., 21-day cycles), e.g., wherein the anti-CD123 immunoconjugate (e.g., pivekimab sunirine) is administered once per cycle. In some aspects, patients can be treated for at least twelve cycles (e.g., 21-day cycles), e.g., wherein the anti-CD123 immunoconjugate (e.g., pivekimab sunirine) is administered once per cycle.

In some aspects, patients can be treated for one to ten cycles (e.g., 21-day cycles), e.g., wherein the anti-CD123 immunoconjugate (e.g., pivekimab sunirine) is administered once per cycle. In some aspects, patients can be treated for two to ten cycles (e.g., 21-day cycles), e.g., wherein the anti-CD123 immunoconjugate (e.g., pivekimab sunirine) is administered once per cycle. In some aspects, patients can be treated for three to ten cycles (e.g., 21-day cycles), e.g., wherein the anti-CD123 immunoconjugate (e.g., pivekimab sunirine) is administered once per cycle. In some aspects, patients can be treated for four to ten cycles (e.g., 21-day cycles), e.g., wherein the anti-CD123 immunoconjugate (e.g., pivekimab sunirine) is administered once per cycle. In some aspects, patients can be treated for five to ten cycles (e.g., 21-day cycles), e.g., wherein the anti-CD123 immunoconjugate (e.g., pivekimab sunirine) is administered once per cycle.

In some aspects, patients can be treated for one to five cycles (e.g., 21-day cycles), e.g., wherein the anti-CD123 immunoconjugate (e.g., pivekimab sunirine) is administered once per cycle. In some aspects, patients can be treated for two to five cycles (e.g., 21-day cycles), e.g., wherein the anti-CD123 immunoconjugate (e.g., pivekimab sunirine) is administered once per cycle. In some aspects, patients can be treated for three to five cycles (e.g., 21-day cycles), e.g., wherein the anti-CD123 immunoconjugate (e.g., pivekimab sunirine) is administered once per cycle.

In certain aspects, about 0.015 mg/kg to about 0.045 mg/kg of an anti-CD123 immunoconjugate (e.g., pivekimab sunirine) is administered, e.g., once in a three-week (21-day) cycle. In some aspects, about 0.015 mg/kg of an anti-CD123 immunoconjugate (e.g., pivekimab sunirine) is administered, e.g., once in a three-week (21-day) cycle. In some aspects, about 0.045 mg/kg of an anti-CD123 immunoconjugate (e.g., pivekimab sunirine) is administered, e.g., once in a three-week (21-day) cycle.

In some aspects, a method provided herein further comprises administering a reduced dose of the anti-CD123 immunoconjugate (e.g., pivekimab sunirine) after a dose-limiting toxicity has occurred in the subject and has been reduced to baseline or <Grade 2.

As further provided herein, an anti-CD123 immunoconjugate (e.g., pivekimab sunirine) can be administered in combination with a BCL-2 inhibitor and/or a HMA. In certain aspects, the anti-CD123 immunoconjugate (e.g., pivekimab sunirine) is administered once in a four-week (28-day) cycle in combination with a BCL-2 inhibitor (e.g. venetoclax) and an HMA (e.g., azacitidine). In some aspects, the anti-CD123 immunoconjugate (e.g., pivekimab sunirine) is administered on Day 7 of the four-week (28-day) cycle.

In certain aspects, about 0.015 mg/kg to about 0.045 mg/kg of an anti-CD123 immunoconjugate (e.g., pivekimab sunirine) is administered, e.g., once in a four-week (28-day) cycle, in combination with a BCL-2 inhibitor (e.g. venetoclax) and an HMA (e.g., azacitidine). In some aspects, about 0.015 mg/kg of an anti-CD123 immunoconjugate (e.g., pivekimab sunirine) is administered, e.g., once in a four-week (28-day) cycle in combination with a BCL-2 inhibitor (e.g. venetoclax) and an HMA (e.g., azacitidine). In some aspects, about 0.045 mg/kg of an anti-CD123 immunoconjugate (e.g., pivekimab sunirine) is administered, e.g., once in a four-week (28-day) cycle in combination with a BCL-2 inhibitor (e.g. venetoclax) and an HMA (e.g., azacitidine). In some aspects, the anti-CD123 immunoconjugate (e.g., pivekimab sunirine) is administered on Day 7 of the four-week (28-day) cycle.

As provided herein, a BCL-2 inhibitor can be administered at a particular dose and/or at particular timing intervals. Administration of a BCL-2 inhibitor (e.g., venetoclax) can be, for example, oral (e.g., in a tablet form).

In certain aspects, a BCL-2 inhibitor (e.g., venetoclax) is administered at a daily dose of up to 400 mg.

In certain aspects, a BCL-2 inhibitor (e.g., venetoclax) is administered in a 28-day cycle. The BCL-2 inhibitor (e.g., venetoclax) can be administered, for example, for 14-28 days of a 28-day cycle. In certain aspects, the BCL-2 inhibitor (e.g., venetoclax) is administered for at least 14 days of a 28-day cycle. In certain aspects, the BCL-2 inhibitor (e.g., venetoclax) is administered for up to 28 days of a 28-day cycle.

In certain aspects, a BCL-2 inhibitor (e.g., venetoclax) is administered orally at a daily dose of up to 400 mg in a 28-day cycle. The BCL-2 inhibitor (e.g., venetoclax) can be administered orally at a daily dose of up to 400 mg, for example, for 14-28 days of a 28-day cycle. In certain aspects, the BCL-2 inhibitor (e.g., venetoclax) is administered orally at a daily dose of up to 400 mg for at least 14 days of a 28-day cycle. In certain aspects, the BCL-2 inhibitor (e.g., venetoclax) is administered orally at a daily dose of up to 400 mg for up to 28 days of a 28-day cycle

As provided herein, an HMA can be administered at a particular dose and/or at particular timing intervals. Administration of an HMA (e.g., azacitidine or decitabine) can be, for example, subcutaneous or intravenous.

In certain aspects, azacitidine can be administered at a daily dose of 75 mg/m² subcutaneously (SC) or intravenously (IV).

In certain aspects, azacitidine can be administered in a 28-day cycle. The azacitidine can be administered, for example, for days 1-7 of a 28-day cycle. In certain aspects, the azacitidine is administered at a dose of at 75 mg/m² subcutaneously (SC) or intravenous (IV) daily for 7 consecutive days, e.g., days 1-7 of a 28-day cycle.

In certain aspects, an HMA (e.g., decitabine) is administered by intravenous infusion over 3 hours, repeated every 8 hours for 3 days, and repeated every six weeks. In certain aspects, an HMA (e.g., decitabine) is administered by intravenous infusion over 1 hour for 5 days and repeated every four weeks.

In certain aspects, 15 mg/m² of an HMA (e.g., decitabine) is administered by intravenous infusion over 3 hours, repeated every 8 hours for 3 days, and repeated every six weeks. In certain aspects, 20 mg/m² of an HMA (e.g., decitabine) is administered by intravenous infusion over 1 hour for 5 days and repeated every four weeks.

In certain aspects, an anti-CD123 immunoconjugate, a BCL-2 inhibitor (e.g., venetoclax), and an HMA (e.g., azacitidine) are administered for one cycle (e.g., a 28-day cycle). In certain aspects, an anti-CD123 immunoconjugate, a BCL-2 inhibitor (e.g., venetoclax), and an HMA (e.g., azacitidine) are administered for repeated cycles, e.g., for 2-12 cycles. In certain aspects, an anti-CD123 immunoconjugate, a BCL-2 inhibitor (e.g., venetoclax), and an HMA (e.g., azacitidine) are administered for two cycles (e.g., 28-day cycles). In certain aspects, an anti-CD123 immunoconjugate, a BCL-2 inhibitor (e.g., venetoclax), and an HMA (e.g., azacitidine) are administered for three cycles (e.g., 28-day cycles). In certain aspects, an anti-CD123 immunoconjugate, a BCL-2 inhibitor (e.g., venetoclax), and an HMA (e.g., azacitidine) are administered for four cycles (e.g., 28-day cycles). In certain aspects, an anti-CD123 immunoconjugate, a BCL-2 inhibitor (e.g., venetoclax), and an HMA (e.g., azacitidine) are administered for five cycles (e.g., 28-day cycles). In certain aspects, an anti-CD123 immunoconjugate, a BCL-2 inhibitor (e.g., venetoclax), and an HMA (e.g., azacitidine) are administered for six cycles (e.g., 28-day cycles). In certain aspects, an anti-CD123 immunoconjugate, a BCL-2 inhibitor (e.g., venetoclax), and an HMA (e.g., azacitidine) are administered for seven cycles (e.g., 28-day cycles). In certain aspects, an anti-CD123 immunoconjugate, a BCL-2 inhibitor (e.g., venetoclax), and an HMA (e.g., azacitidine) are administered for eight cycles (e.g., 28-day cycles). In certain aspects, an anti-CD123 immunoconjugate, a BCL-2 inhibitor (e.g., venetoclax), and an HMA (e.g., azacitidine) are administered for nine cycles (e.g., 28-day cycles). In certain aspects, an anti-CD123 immunoconjugate, a BCL-2 inhibitor (e.g., venetoclax), and an HMA (e.g., azacitidine) are administered for ten cycles (e.g., 28-day cycles). In certain aspects, an anti-CD123 immunoconjugate, a BCL-2 inhibitor (e.g., venetoclax), and an HMA (e.g., azacitidine) are administered for eleven cycles (e.g., 28-day cycles). In certain aspects, an anti-CD123 immunoconjugate, a BCL-2 inhibitor (e.g., venetoclax), and an HMA (e.g., azacitidine) are administered for twelve cycles (e.g., 28-day cycles).

In certain aspects, an anti-CD123 immunoconjugate, a BCL-2 inhibitor (e.g., venetoclax), and an HMA (e.g., azacitidine) are administered for one to twelve cycles (e.g., 28-day cycles). In certain aspects, an anti-CD123 immunoconjugate, a BCL-2 inhibitor (e.g., venetoclax), and an HMA (e.g., azacitidine) are administered for one to ten cycles (e.g., 28-day cycles). In certain aspects, an anti-CD123 immunoconjugate, a BCL-2 inhibitor (e.g., venetoclax), and an HMA (e.g., azacitidine) are administered for one to eight cycles (e.g., 28-day cycles). In certain aspects, an anti-CD123 immunoconjugate, a BCL-2 inhibitor (e.g., venetoclax), and an HMA (e.g., azacitidine) are administered for one to six cycles (e.g., 28-day cycles).

In certain aspects, an anti-CD123 immunoconjugate, a BCL-2 inhibitor (e.g., venetoclax), and an HMA (e.g., azacitidine) are administered for two to twelve cycles (e.g., 28-day cycles). In certain aspects, an anti-CD123 immunoconjugate, a BCL-2 inhibitor (e.g., venetoclax), and an HMA (e.g., azacitidine) are administered for two to ten cycles (e.g., 28-day cycles). In certain aspects, an anti-CD123 immunoconjugate, a BCL-2 inhibitor (e.g., venetoclax), and an HMA (e.g., azacitidine) are administered for two to eight cycles (e.g., 28-day cycles). In certain aspects, an anti-CD123 immunoconjugate, a BCL-2 inhibitor (e.g., venetoclax), and an HMA (e.g., azacitidine) are administered for two to six cycles (e.g., 28-day cycles).

### V(C). Clinical Outcomes Achieved by Anti-CD123 Immunoconjugates

As demonstrated herein, administration of an anti-CD123 immunoconjugate such as pivekimab sunirine, alone or in combination with a BCL-2 inhibitor and/or an HMA can achieve a clinical benefit in a patient.

As also demonstrated herein, administration of an anti-CD123 immunoconjugate such as pivekimab sunirine, alone or in combination with a BCL-2 inhibitor and/or an HMA, can achieve bone marrow remission in a subject with AML with bone marrow involvement.

As also demonstrated herein, administration of an anti-CD123 immunoconjugate such as pivekimab sunirine, alone or in combination with a BCL-2 inhibitor and/or an HMA, can produce a complete response (CR), CR (clinical) with partial hematological recovery (CRh), or CR (clinical) with incomplete recovery (CRi) in a subject with AML. In some aspects, the time to the CR, CRh, or CRi is about 3 months or less. In some aspects, the time to the CR, CRh, or CRi is about 2.5 months or less. I

In some aspects, the time to the CR, CRh, or CRi is about 0.5 to about 3 months. In some aspects, the time to the CR, CRh, or CRi is about 0.5 to about 2.5 months.

In some aspects, the time to the CR, CRh, or CRi is about 1 to about 3 months. In some aspects, the time to the CR, CRh, or CRi is about 1 to about 2.5 months.

In some aspects, the duration of the CR, CRh, or CRi is at least 2 months. In some aspects, the duration of the CR, CRh, or CRi is at least 3 months.

In some aspects, administration of an anti-CD123 immunoconjugate such as pivekimab sunirine, alone or in combination with a BCL-2 inhibitor and/or an HMA, produces a CR, CRh, or CRi after 3 or less doses of the anti-CD123 immunoconjugate. In some aspects, administration of an anti-CD123 immunoconjugate such as pivekimab sunirine, alone or in combination with a BCL-2 inhibitor and/or an HMA, produces a CR, CRh, or CRi after 1 to 3 doses of the anti-CD123 immunoconjugate. In some aspects, administration of an anti-CD123 immunoconjugate such as pivekimab sunirine, alone or in combination with a BCL-2 inhibitor and/or an HMA, produces a CR, CRh, or CRi after 1 or 2 doses of the anti-CD123 immunoconjugate.

In some aspects, a patient with AML is eligible for HSCT after achieving a CR, CRh, or CRi in response to treatment with an anti-CD123 immunoconjugate such as pivekimab sunirine, alone or in combination with a BCL-2 inhibitor and/or an HMA.

As also demonstrated herein, administration of an anti-CD123 immunoconjugate such as pivekimab sunirine, alone or in combination with a BCL-2 inhibitor and/or an HMA, can prepare a subject for a hematopoietic stem cell transplant (HSCT).

In some aspects, a patient with AML is eligible for HSCT after 1 to 3 doses of an anti-CD123 immunoconjugate such as pivekimab sunirine, alone or in combination with a BCL-2 inhibitor and/or an HMA. In some aspects, a patient with AML is eligible for HSCT after 1 or 2 doses of an anti-CD123 immunoconjugate such as pivekimab sunirine, alone or in combination with a BCL-2 inhibitor and/or an HMA.

As also demonstrated herein, administration of an anti-CD123 immunoconjugate such as pivekimab sunirine in combination with a BCL-2 inhibitor and/or an HMA, can clear minimal residual disease in a subject within 2 months or less.

As also demonstrated herein, administration of an anti-CD123 immunoconjugate such as pivekimab sunirine in combination with a BCL-2 inhibitor and/or an HMA, can achieve CR, CRh, CRp, or CRi with a duration of at least 3 months, at least 4 months, at least 5 months, at least 6 months, at least 10 months, or at least 12 months.

As also demonstrated herein, administration of an anti-CD123 immunoconjugate such as pivekimab sunirine in combination with a BCL-2 inhibitor and/or an HMA can achieve an absolute neutrophil count (ANC) recovery to ≥500/μL (microliter) in a subject within 35 days.

As also demonstrated herein, administration of an anti-CD123 immunoconjugate such as pivekimab sunirine in combination with a BCL-2 inhibitor and/or an HMA can achieve a platelet count recovery to >50,000/L (microliter) in a subject within 25 days.

### V(D). Premedications

In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with a premedication. In some embodiments, the premedication may be a corticosteroid. Accordingly, in some aspects, the methods provided herein comprise administering a corticosteroid to a patient prior to administering an anti-CD123 immunoconjugate (e.g., pivekimab sunirine) to the patient.

In some embodiments, the corticosteroid may be administered once, prior to and on the same day as the pivekimab sunirine. In some aspects, the corticosteroid is administered to a patient on the day prior to administering an anti-CD123 immunoconjugate, e.g., pivekimab sunirine, to the patient. In some aspects, the corticosteroid is administered to a patient on the day prior and once, prior to and on the same day as administering an anti-CD123 immunoconjugate, e.g., pivekimab sunirine, to the patient. Accordingly, in some aspects, the corticosteroid is administered to a patient twice on the day prior to administering an anti-CD123 immunoconjugate, e.g., pivekimab sunirine, to the patient. For example, in some aspects, a patient receives two doses of a corticosteroid on the day prior to administering an anti-CD123 immunoconjugate, e.g., pivekimab sunirine. In some embodiments, the corticosteroid may be administered twice on the day prior to administration of the pivekimab sunirine and once on the same day as the pivekimab sunirine, where all administrations of the corticosteroid are administered prior to the pivekimab sunirine. In embodiments, the premedication dosing may provide for less infusion related reactions.

In certain instances, the corticosteroid can be selected from the group consisting of prednisone, prednisolone, methylprednisolone, beclomethasone, betamethasone, dexamethasone, fludrocortisone, hydrocortisone, and triamcinolone. In certain instances the corticosteroid is administered intravenously. In certain instances, the steroid is administered orally.

For example, in some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with diphenhydramine. In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with 25-50 mg diphenhydramine. In some aspects, diphenhydramine is given intravenously. In some aspects, diphenhydramine is given orally. Accordingly, in some aspects, the methods provided herein comprise administering diphenhydramine to a patient prior to administering an anti-CD123 immunoconjugate (e.g., pivekimab sunirine) to the patient.

In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with acetaminophen. In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with 325-650 mg acetaminophen. In some aspects, acetaminophen is given intravenously. In some aspects, acetaminophen is given orally. Accordingly, in some aspects, the methods provided herein comprise administering acetaminophen to a patient prior to administering an anti-CD123 immunoconjugate (e.g., pivekimab sunirine) to the patient.

In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with paracetamol. In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with 325-650 mg paracetamol. In some aspects, paracetamol is given intravenously. In some aspects, paracetamol is given orally. Accordingly, in some aspects, the methods provided herein comprise administering paracetamol to a patient prior to administering an anti-CD123 immunoconjugate (e.g., pivekimab sunirine) to the patient.

In some aspects, patients receiving an anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with dexamethasone. In some aspects, patients receiving anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with 8 mg dexamethasone. In some aspects, patients receiving anti-CD123 immunoconjugate (e.g., pivekimab sunirine), alone or in combination with a BCL-2 inhibitor (e.g., venetoclax) and/or a hypomethylating agent (HMA) (e.g., azacitidine or decitabine), as disclosed herein have received pretreatment with 10 mg dexamethasone. In some aspects, dexamethasone is given intravenously. In some aspects, dexamethasone is given orally. Accordingly, in some aspects, the methods provided herein comprise administering dexamethasone to a patient prior to administering an anti-CD123 immunoconjugate (e.g., pivekimab sunirine) to the patient. Accordingly, in some aspects, the dexamethasone is administered to a patient the day prior to administering an anti-CD123 immunoconjugate, e.g., pivekimab sunirine, to the patient. Accordingly, in some aspects, the dexamethasone is administered to a patient twice on the day prior to administering an anti-CD123 immunoconjugate, e.g., pivekimab sunirine, to the patient. For example, in some aspects, a patient receives two doses of dexamethasone (e.g., two 8 mg doses; two 10 mg doses) on the day prior to administering an anti-CD123 immunoconjugate, e.g., pivekimab sunirine.

In some aspects, patients receiving an anti-CD123 immunoconjugate disclosed herein, e.g., pivekimab sunirine, also receive a CNS prophylactic treatment. In some aspects, the CNS prophylactic treatment comprises intrathecal chemotherapy.

In some aspects, patients receiving an anti-CD123 immunoconjugate disclosed herein, e.g., pivekimab sunirine, also receive ursodeoxycholic acid.

In some embodiments (E1 to E115), the disclosure provides:
E1. A method of treating an unfit newly diagnosed acute myeloid leukemia (ND AML) in a subject comprising administering to the subject a combination therapy comprising (i) pivekimab sunirine, (ii) a BCL-2 inhibitor, and (iii) a hypomethylating agent.
E2. A method for treating an acute myeloid leukemia (AML) in a subject comprising administering to the subject a combination therapy comprising (i) pivekimab sunirine, (ii) a BCL-2 inhibitor, and (iii) a hypomethylating agent, wherein the administration achieves a complete response (CR), CR (clinical) with partial hematological recovery (CRh), CR with incomplete platelet recovery (CRp), or CR (clinical) with incomplete recovery (CRi) with a duration of at least 3 months.
E3. A method for treating a FLT3 TKD, IDH1^{mut}, NMP1^{mut}, and/or K/NAS^{mut} acute myeloid leukemia (AML) in a subject with comprising administering to the subject a combination therapy comprising (i) pivekimab sunirine, (ii) a BCL-2 inhibitor, and (iii) a hypomethylating agent.
E4. A method of clearing minimal residual disease (MRD) in a human subject with acute myeloid leukemia (AML) comprising administering to the subject a combination therapy comprising (i) pivekimab sunirine, (ii) a BCL-2 inhibitor, and (iii) a hypomethylating agent, wherein the MRD is cleared in 2 months or less.
E5. The method of any one of E1, E3, and E4, wherein the administration of the combination therapy achieves a complete response with a duration of at least 3 months.
E6. The method of E2 or E5, wherein the duration of the CR, CRh, CRp, or CRi is at least 4 months.
E7. The method of E2 or E5, wherein the duration of the CR, CRh, CRp, or CRi is at least 5 months or at least 6 months.
E8. The method of E2 or E5, wherein the duration of the CR, CRh, CRp, or CRi is at least 10 months or at least 12 months.
E9. The method of any one of E2-E8, wherein the AML is unfit ND AML.
E10. The method of any one of E1, E2, and E4-E9, wherein the ND AML is a FLT3 TKD, IDH1^{mut}, NMP1^{mut}, and/or K/NAS^{mut} AML.
E11. The method of any one of E1-E3 and E5-E10, wherein the administration of the combination therapy achieves a minimal residual disease (MRD)-negative status in 2 months or less.
E12. The method of any one of E1-E11, wherein the method further comprises administering a hematopoietic stem cell transplant (HSCT) to the subject.
E13. The method of E12, wherein the HSCT is allogeneic.
E14. The method of E12, wherein the HSCT is autologous.
E15. The method of any one of E12-E14, wherein the HSCT is administered about 4 to about 8 weeks after the administration of pivekimab sunirine.
E16. A method of preparing a subject with acute myeloid leukemia (AML) for a hematopoietic stem cell transplant (HSCT), the method comprising administering to the subject a combination therapy comprising (i) pivekimab sunirine, (ii) a BCL-2 inhibitor, and (iii) a hypomethylating agent.
E17. A method for treating an acute myeloid leukemia (AML) in a subject comprising (i) administering to the subject a combination therapy comprising (i) pivekimab sunirine, (ii) a BCL-2 inhibitor, and (iii) a hypomethylating agent and (ii) subsequently administering a hematopoietic stem cell transplant (HSCT) to the subject.
E18. A method for treating an acute myeloid leukemia (AML) in a subject comprising administering a hematopoietic stem cell transplant (HSCT) to the subject, wherein the subject has previously been treated with pivekimab sunirine.
E19. The method of any one of E16-E18, wherein the HSCT is allogeneic.
E20. The method of any one of E16-E18, wherein the HSCT is autologous.
E21. The method of any one of E16-E20, wherein the HSCT is administered about 4 to about 8 weeks after the administration of pivekimab sunirine.
E22. The method of any one of E16-E21, wherein the administration achieves a minimal residual disease (MRD)-negative status.
E23. The method of any one of E16-E22, wherein the AML is a FLT3 TKD, IDH1^{mut}, NMP1^{mut}, and/or K/NAS^{mut} AML.
E24. The method of any one of E16-E23, wherein the pivekimab sunirine is administered once every three weeks (Q3W).
E25. The method of any one of E1-E24, wherein the administration of the combination therapy results in survival for at least 3 months.
E26. The method of any one of E1-E24, wherein the administration of the combination therapy results in survival for at least 6 months.
E27. The method of any one of E1-E26, wherein the administration of the combination therapy achieves undetectable disease.
E28. The method of any one of E1-E27, wherein the administration of the combination therapy results in a pivekimab sunirine Cmax of about 400 to about 600 ng/mL after a single administration of the pivekimab sunirine and/or a pivekimab sunirine Cmax of about 550 to 700 ng/mL after three administrations of the pivekimab sunirine.
E29. The method of any one of E1-E28, wherein the administration of the combination therapy results in a pivekimab sunirine AUC_{INF} of about 4,500 to about 5,000 h*ng/mL after a single administration of the pivekimab sunirine and/or a pivekimab sunirine AUC_{INF} of about 4,750 to about 5,250 h*ng/mL after three administrations of the pivekimab sunirine.
E30. The method of any one of E1-E29, wherein the administration of the combination therapy results in no more than 30% CD123 receptor availability 4 hours after administration of the pivekimab sunirine.
E31. The method of any one of E1-E30, wherein the AML is a TP53^{WT} AML.
E32. The method of E31, wherein the AML is a K/NRAS WT AML with no FLT3-ITD.
E33. The method of E31, wherein the AML is a FLT3-ITD or K/NRAS^{mut} AML.
E34. The method of any one of E1-E30, wherein the AML is a TP53^{mut} AML.
E35. The method of any one of E1-E30, wherein the AML is a FLT3 ITD or TKD, IDH1^{mut}, IDH2^{mut}, NPM1^{mut}, and/or K/NRAS^{mut} AML.
E36. The method of any one of E1-E30, wherein the subject has a baseline mutation in RUNX1, ASXL1, or TP53.
E37. The method of any one of E1-E36, wherein the AML has a favorable ELN 2017 risk.
E38. The method of any one of E1-E36, wherein the AML has an intermediate ELN 2017 risk.
E39. The method of any one of E1-E36, wherein the AML has an adverse ELN 2017 risk.
E40. The method of any one of E1-E39, wherein the subject was previously treated with an anti-CD123 therapy, optionally wherein the anti-CD123 therapy was tagraxofusp (SL-401).
E41. The method of any one of E1-E39, wherein the subject has not received tagraxofusp (SL-401) prior to the administration of the combination therapy.
E42. The method of any one of E1-E41, wherein the AML expresses multidrug resistance 1 (MDR1).
E43. The method of any one of E1-E42, wherein the AML expresses P-glycoprotein (P-gp).
E44. The method of any one of E1-E43, wherein the subject has an absolute neutrophil count of greater than 500/μL (microliter).
E45. The method of any one of E2, E3-E8, and E10-E44, wherein the AML is unfit AML.
E46. The method of any one of E2, E3-E8, and E10-E44, wherein the AML is fit AML.
E47. The method of any one of E1-E46, wherein the cancer has previously been treated with venetoclax.
E48. The method of any one of E1-E46, wherein the cancer has not previously been treated with venetoclax.
E49. The method of any one of E1-E48, wherein the cancer has previously been treated with a hypomethylating agent.
E50. The method of any one of E1-E48, wherein the cancer has not previously been treated with a hypomethylating agent.
E51. The method of any one of E1-E50, wherein the subject received a stem cell transplant prior to the administration of the combination therapy.
E52. The method of E51, wherein the stem cell transplant was received at least 120 days prior to the administration.
E53. The method of E51 or E52, wherein the previous stem cell transplant was allogeneic.
E54. The method of E51 or E52, wherein the previous stem cell transplant was autologous.
E55. The method of any one of E16-E54, wherein the AML is a relapsed and/or refractory AML.
E56. The method of E55, wherein the AML is a relapsed AML.
E57. The method of E55, wherein the AML is a refractory AML.
E58. The method of any one of E16-E57, wherein the subject received at least one prior line of therapy, at least two prior lines of therapy, or at least three prior lines of therapy.
E59. The method of any one of E1-E15, and E25-E54 wherein the administration of the combination therapy is a frontline therapy.
E60. The method of any one of E1-E59, wherein the AML is de novo AML.
E61. The method of any one of E1-E59, wherein the subject has a prior or concomitant hematologic malignancy (PCHM).
E62. The method of E61, wherein the PCHM does not require immediate therapy.
E63. The method of E61 or E62, wherein the PCHM is in remission and the subject has completed all therapies for the PCHM at least 6 months prior to the administration of the combination therapy.
E64. The method of any one of E1-E63, wherein the subject has not received immunosuppressive treatment for at least 14 days prior to the administration of the combination therapy.
E65. The method of any one of E1-E64, wherein the subject has not received a systemic anti-cancer therapy for at least 14 days prior to the administration of the combination therapy.
E66. The method of any one of E1-E64, wherein the subject has received a local therapy at least 14 days prior to the administration of the combination therapy.
E67. The method of E66, wherein the therapy was radiotherapy.
E68. The method of any one of E1-E67, wherein the method further comprises administration of ursodeoxycholic acid.
E69. The method of any one of E1-E68, wherein the subject has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.
E70. The method of any one of E1-E69, wherein the pivekimab sunirine is administered intravenously.
E71. The method of any one of E1-E70, wherein the pivekimab sunirine is administered by a controlled rate of infusion.
E72. The method of any one of E1-E71, wherein the pivekimab sunirine is administered in an infusion having a duration of no more than 30 minutes.
E73. The method of any one of E1-E72, wherein the pivekimab sunirine is administered in an infusion having a duration of about 15 to 30 minutes.
E74. The method of any one of E71-E73, wherein the controlled rate of infusion is from about 0.8 mL/min to about 1.7 mL/min.
E75. The method of any one of E71-E73, wherein the controlled rate of infusion is about 0.8 m/min (about 50 mL/hour or about 0.165 mg/min) for the first 30 minutes.
E76. The method of any one of E71-E74, wherein the controlled rate of infusion is increased to about 1.7 m/min (about 100 mL/hour or about 0.33 mg/min).
E77. The method of any one of E1-E76, wherein the pivekimab sunirine is administered at a dose of about 0.045 mg/kg.
E78. The method of any one of E1-E15, wherein the pivekimab sunirine is administered once every 4 weeks (Q4W).
E79. The method of any one of E1-15 or E78, wherein the BCL-2 inhibitor is administered daily for at least 14 days of a 28-day cycle.
E80. The method of any one of E1-15 or E78, wherein the BCL-2 inhibitor is administered daily for up to 28 days of a 28-day cycle.
E81. The method of any one of E1-15 or E78-E80, wherein the BCL-2 inhibitor is administered at a dose of up to about 400 mg.
E82. The method of any one of E1-15 or E78-E81, wherein the BCL-2 inhibitor is administered orally.
E83. The method of any one of E1-15 or E78-E82, wherein the BCL-2 inhibitor is venetoclax.
E84. The method of any one of E1-E15 or E78-E83, wherein the hypomethylating agent is administered daily for days 1-7 of a 28-day cycle.
E85. The method of any one of E1-E15 or E78-E84, wherein the hypomethylating agent is administered at a dose of about 75 mg/m².
E86. The method of any one of E1-E15 or E78-E85, wherein the hypomethylating agent is administered subcutaneously or intravenously.
E87. The method of any one of E1-E15 or E78-E86, wherein the hypomethylating agent is azacitidine.
E88. The method of any one of E1-E15 or E78-E86, wherein the hypomethylating agent is decitabine.
E89. The method of any one of E1-E15 or E78-E88, wherein the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for at least 14 days.
E90. The method of any one of E1-E15 or E78-E88, wherein the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for up to 28 days.
E91. The method of any one of E1-E90, wherein the administration is for one cycle.
E92. The method of any one of E1-E90, wherein the administration is for more than one cycle, optionally wherein the administration is for at least 2 cycles, at least 3 cycles, at least 4 cycles, at least 5 cycles, at least 6 cycles, at least 7 cycles, at least 8 cycles, at least 9 cycles, or at least 10 cycles or wherein the administration is for about 2-4 cycles, about 2-6 cycles, about 2-8 cycles, about 2-10 cycles, or about 2-12 cycles.
E93. The method of any one of E1-E92, wherein the method further comprises administering a reduced dose of the pivekimab sunirine after a dose-limiting toxicity has occurred in the subject and has been reduced to baseline or <Grade 2.
E94. The method of any one of E1-E93, wherein the AML is CD123-positive.
E95. The method of any one of E1-E94, wherein CD123 has been detected in a sample obtained from the AML prior to the administration of the combination therapy, optionally wherein the CD123 was detected using flow cytometry or immunohistochemistry.
E96. The method of any one of E1-E95, further comprising detecting CD123 in a sample obtained from the AML prior to the administration of the combination therapy.
E97. The method of any one of E1-E96, wherein at least 80% of cells in the AML express CD123.
E98. The method of any one of E1-E97, wherein CD123 has been detected in at least 80% of cells in a sample obtained from the AML prior to the administration of the combination therapy.
E99. The method of any one of E1-E98, further comprising detecting CD123 in at least 80% of cells in a sample obtained from the AML prior to the administration of the combination therapy.
E100. The method of any one of E1-E99, wherein the subject is not selected based on level of CD123 expression.
E101. The method of any one of E1-E100, wherein the subject has been pretreated with a premedication or corticosteroid prior to administration of the pivekimab sunirine, optionally wherein the premedication or corticosteroid is diphenhydramine, acetaminophen, paracetamol, dexamethasone, or a combination thereof.
E102. The method of any one of E1-E100, further comprising pre-treating the subject with a premedication or corticosteroid prior to administration of the pivekimab sunirine, optionally wherein the premedication or corticosteroid is diphenhydramine, acetaminophen, paracetamol, dexamethasone, or a combination thereof.
E103. The method of any one of E101-E102, wherein the corticosteroid is administered to a patient on the day prior and once, prior to and on the same day as administering an anti-CD123 immunoconjugate, e.g., pivekimab sunirine, to the patient.
E104. The method of any one of E101-E102, wherein the corticosteroid is administered to a patient twice on the day prior and once, prior to and on the same day as administering an anti-CD123 immunoconjugate, e.g., pivekimab sunirine, to the patient.
E105. The method of any one of E101-E104, wherein the corticosteroid is dexamethasone.
E106. The method of E105, wherein the dexamethasone is administered at a dose of 8 mg or 10 mg.
E107. The method of any one of E101-E106, wherein the subject is further premedicated with an antihistamine and an antipyretic at least 30 minutes prior to dosing with pivekimab sunirine anti-CD123 immunoconjugate.
E108. The method of E107, wherein the antihistamine is diphenhydramine and the antipyretic is acetaminophen or paracetamol.
E109. The method of E108, wherein diphenhydramine is administered intravenously at a dose of 25 mg to 50 mg; and (i) acetaminophen is administered orally or intravenously at a dose of 325 mg to 650 mg or (ii) paracetamol is administered orally or intravenously at a dose of 500 mg to 1000 mg.
E110. A method of treating an unfit newly diagnosed acute myeloid leukemia (ND AML) in a subject comprising administering to the subject (i) 0.045 mg/kg pivekimab sunirine, (ii) venetoclax, and (iii) azacitidine, wherein the administration achieves a complete response (CR), CR (clinical) with partial hematological recovery (CRh), CR with incomplete platelet recovery (CRp), or CR (clinical) with incomplete recovery (CRi) with a duration of at least 3 months.
E111. The method of E110, wherein the AML is a FLT3 TKD, IDH1^{mut}, NMP1^{mut}, and/or K/NAS^{mut} AML.
E112. The method of E110 or E111, wherein the pivekimab sunirine is administered on day 7 of a 28 day cycle; the is azacitidine and it is administered at a dose of 75 mg/m² on days 1-7 of the cycle, and the venetoclax is administered at a dose of 400 mg for at least 14 days of the cycle.
E113. The method of E110 or E111, wherein the pivekimab sunirine is administered on day 7 of a 28 day cycle; the is azacitidine and it is administered at a dose of 75 mg/m² on days 1-7 of the cycle, and the venetoclax is administered at a dose of 400 mg for up to 28 days of the cycle.
E114. A method of treating relapsed and/or refractory AML in a subject comprising administering to the subject (i) 0.045 mg/kg pivekimab sunirine, (ii) venetoclax, and (iii) azacitidine, wherein the administration achieves a complete response (CR), CR (clinical) with partial hematological recovery (CRh) or CR (clinical) with incomplete recovery (CRi) complete response with a duration of at least 2 months.
E115. Use of pivekimab sunirine in the method of any one of E1-E114 or in the preparation for a medicament for use in the method of any one of E1-E114.

Aspects of the present disclosure can be further defined by reference to the following non-limiting examples. It will be apparent to those skilled in the art that many modifications, both to materials and methods, can be practiced without departing from the scope of the present disclosure.

### EXAMPLES

It is understood that the examples and aspects described herein are for illustrative purposes only and that various modifications or changes in light thereof will be suggested to persons skilled in the art and are to be included within the spirit and purview of this application

### Example 1: Treatment of AML in Human Patients Using Pivekimab Sunirine

Patients with AML were enrolled into a phase 1/2 clinical trial of pivekimab sunirine. Key inclusion and exclusion criteria were as follows.

### Key Inclusion Criteria

Patients ≥18 years old
R/R AML
Confirmation of CD123 positivity on flow cytometry or immunohistochemistry (IHC). Patients who received prior CD123-targeting agents were allowed as long as the blasts still had detectable CD123 expression.
Eastern Cooperative Oncology Group (ECOG) performance status ≤1. If nonambulatory due to a chronic disability, Karnofsky performance status was >70.
Previous treatment-related toxicities must have been resolved to Grade 1 (excluding alopecia).
Liver enzymes (aspartate aminotransferase and alanine aminotransferase)≤2.5× the upper limit of normal (ULN). Exceptions could be made for patients with elevated liver transaminases secondary to the underlying study disease.
Total bilirubin ≤1.5×ULN; patients with Gilbert syndrome had have total bilirubin <3.0×ULN with direct bilirubin <1.0×ULN at the time of enrollment.
Estimated glomerular filtration rate of >30 mL/min/1.73 m² or creatinine clearance of >30 m/min.
Left ventricular ejection fraction >45%.
Patients with a prior autologous or allogeneic bone marrow transplant were eligible for Cohorts 1 and 2. Patients with an allogeneic transplant met the following conditions: the transplant must have been performed more than 120 days before the date of dosing on this study, the patient must not have active ≥Grade 2 acute graft versus host disease (GvHD), or extensive chronic GvHD of any severity, and must be off all immunosuppression for at least 2 weeks before first dose of pivekimab sunirine.
Patients with prior malignancy were eligible. Patients with a PCHM were eligible as long as no current therapy was still required for the second malignancy (e.g., MDS, CMML). Patients with a nonhematologic prior malignancy were in remission from the prior malignancy. Patients had completed all chemotherapy and radiotherapy for prior malignancy at least 6 months before enrollment and all treatment-related toxicities must have resolved to Grade 1 or less.
Patients with prostate cancer or breast cancer on adjuvant hormonal therapy were eligible.
Patients with tumors with a negligible risk for metastasis or death (e.g., adequately controlled basal cell carcinoma or squamous cell carcinoma of the skin, or carcinoma in situ of the cervix or breast) were eligible.

### Key Exclusion Criteria

Patients with a history of veno-occlusive disease of the liver.
Patients with a history of Grade 4 capillary leak syndrome, or noncardiac Grade 4 edema are ineligible, e.g., related to tagraxofusp-erzs or other etiology.
Corrected QT interval (QT interval corrected using Fridericia's formula) >480 msec.
Myocardial infarction within 6 months before enrollment or has New York Heart Association Class III or IV heart failure, uncontrolled angina, severe uncontrolled ventricular arrhythmias, or electrocardiographic evidence of acute ischemia or active conduction system abnormalities before study entry.
Interval from prior cancer therapy:
   Patients who have received a checkpoint inhibitor must not have received that therapy within 28 days before drug administration on this study.
Clinically relevant active infection including known active hepatitis B or C, human immunodeficiency virus infection, or cytomegalovirus or any other known concurrent infectious disease that, in the judgment of the Investigator, would make a patient inappropriate for enrollment into this study (testing not required).
Patients who have undergone a major surgery within 4 weeks (or longer if not fully recovered) before study enrollment.
Serious or poorly controlled medical conditions that could be exacerbated by treatment or that would seriously compromise safety assessment or compliance with the protocol, in the judgment of the Investigator.
Patients who have a history of allergy to pivekimab sunirine or any of its excipients.
Patients who received a live vaccine four weeks or fewer prior to enrollment.

Patients with R/R AML (n=68) were treated with pivekimab sunirine once every 3 weeks (Q3W; on day 1 of a 3-week cycle). The characteristics of these patients are listed in Table 4.

TABLE 4
AML Patient Characteristics

| | All Participants in Escalation | |
|---|---|---|
| | and Expansion Phases (n = 68) | |
| Age, median (range), y | 68 | (31-83) |
| ≥65 y of age, No. (%) | 43 | (63) |
| Male, No. (%) | 38 | (56) |
| History/type of AML, No. (%) | | |
| De novo AML | 39 | (57) |
| Secondary AML (including therapy- | 29 | (43) |
| related AML) | | |
| ELN 2017 risk category, No. (%) | | |
| Favourable | 3 | (4) |
| Intermediate | 20 | (29) |
| Adverse | 33 | (49) |
| Not determined/missing | 12 | (18) |
| Prior therapy, No. (%) | | |
| Nonintensive | 50 | (74) |
| Intensive | 53 | (78) |
| SCT | 21 | (31) |
| Radiotherapy | 2 | (3) |
| Prior systemic therapy regimens, | | |
| No. (%) | | |
| Median (range) | 2 | (1-4) |
| 1 | 19 | (28) |
| 2 | 33 | (49) |
| 3 | 14 | (21) |
| 4 | 2 | (3) |
| Disease status, No. (%) | | |
| Primary refractory | 11 | (16) |
| First relapse | 26 | (38) |
| Relapsed/refractory | 31 | (46) |
| Common mutations, No. (%) | | |
| FLT3 ITD/TKD | 5 | (7) |
| TP53 | 8 | (12) |

The trial included a dose-escalation phase and a dose-expansion phase. All patients in both phases received intravenous doses of pivekimab sunirine on day 1 of a 21-day cycle as a <30-minute outpatient infusion. Lyophilized pivekimab sunirine was reconstituted using 5.4 mFL of water for injection. Planned treatment consisted of 2 cycles (6 weeks). Additional cycles, up to 10 total, were administered for patients deriving benefit from this regimen. CNS prophylactic intrathecal chemotherapy was administered. Patients received 8 mg dexamethasone on the day before any pivekimab sunirine dose. Patients also received 25-50 mg diphenhydramine, 325-650 mg acetaminophen or paracetamol, and 8 mg dexamethasone before each pivekimab sunirine infusion.

Following preparation, IMGN632 (or pivekimab sunirine (pvek)) was administered by IV syringe pump. Overall length of infusion was varied depending on dose and patient tolerability. Initially, the study drug was administered at a rate of 0.165 mg/min (0.8 m/min); and, after 30 minutes, if well tolerated, the rate was increased to 0.33 mg/min. Subsequent infusions were delivered at the highest tolerated rate. Before and following infusion, the line was flushed with 5% dextrose to ensure delivery of the full dose. Patients were carefully observed during each infusion and vital signs were monitored. Patients remained in the clinic under observation for 4 hours after the first infusion and for at least 1 hour after each subsequent infusion.

In the dose-escalation phase, patients received doses of pivekimab sunirine starting at 0.015 mg/kg with five escalations (n=38): 0.015 mg/kg (n=3), 0.045 mg/kg (n=9), 0.09 mg/kg (n=12), 0.018 mg/kg (n=7), 0.3 mg/kg (n=5), and 0.45 mg/kg (n=2).

Based on nonclinical pharmacology, clinical safety, anti-leukemia activity, PK, and PD assessments during the dose-escalation phase, two cohorts were established for the dose expansion phase.
Cohort 1: Patients with relapsed AML received 0.045 mg/kg (escalation dose level 2) Q3W (n=20).
Cohort 2: Patients with relapsed or refractory (to non-intense therapies) AML received 0.09 mg/kg (escalation dose level 3) Q3W (n=10).

A dose of 0.045 mg/kg pivekimab sunirine Q3W was established as a biologically effective dose and selected as the recommended phase 2 dose (RP2D) based on the favorable safety and similar anti-leukemia activity in higher doses, PK, and PD information as detailed below.

Safety was assessed by treatment-emergent adverse events (TEAEs), serious adverse events (SAEs), and dose-limiting toxicities (DLTs). The median duration of treatment in participants who received 0-045 mg/kg pivekimab sunirine Q3W (n=29) was 9 weeks (range, 3-110).

TEAEs occurred in 28 RP2D-treated participants (97%) (Table 5). TEAEs (all grades) occurring in ≥20% of RP2D-treated participants were epistaxis (n=9; 31%), febrile neutropenia (n=8; 28%), diarrhea (n=8; 28%), fatigue (n=8; 28%), constipation (n=7; 24%), peripheral edema (n=7; 24%), pneumonia (n=6; 21%), nausea (n=6; 21%), pyrexia (n=6; 21%), and chills (n=6; 21%). The most common (≥10%) grade ≥3 TEAEs in the RP2D-treated participants were febrile neutropenia (n=8; 28%), pneumonia (n=5; 17%), anemia (n=5; 17%), sepsis (n=3; 10%), thrombocytopenia (n=3; 10%), and leukocytosis (n=3; 10%).

TABLE 5
TEAEs Occurring in ≥10% of Patients Receiving
0.045 mg/kg pivekimab sunirine Q3W

| | |
|---|---|
| | TEAEs at Recommended Phase 2 Dose |
| Adverse Event, No. | (RP2D) (n = 29) |

| (%) | All Grades | Grade ≥3 |
|---|---|---|
| Any event | 28 (97) | 22 (76) |
| Epistaxis | 9 (31) | 1 (3) |
| Febrile neutropenia | 8 (28) | 8 (28) |
| Diarrhoea | 8 (28) | 0 |
| Fatigue | 8 (28) | 1 (3) |
| Constipation | 7 (24) | 0 |
| Peripheral oedema | 7 (24) | 0 |
| Pneumonia | 6 (21) | 5 (17) |
| Nausea | 6 (21) | 1 (3) |
| Pyrexia | 6 (21) | 0 |
| Chills | 6 (21) | 0 |
| IRR | 5 (17) | 2 (7) |
| Anaemia | 5 (17) | 5 (17) |
| Cellulitis | 5 (17) | 2 (7) |
| Petechiae | 5 (17) | 0 |
| Cough | 4 (14) | 0 |
| ALT increase | 4 (14) | 1 (3) |
| Headache | 4 (14) | 0 |
| Insomnia | 4 (14) | 0 |
| Vomiting | 4 (14) | 1 (3) |
| Pain in extremity | 4 (14) | 2 (7) |
| Decreased appetite | 3 (10) | 0 |
| Abdominal pain | 3 (10) | 0 |
| Dyspnoea | 3 (10) | 1 (3) |
| Hyperglycaemia | 3 (10) | 0 |
| Hypotension | 3 (10) | 0 |
| Sepsis | 3 (10) | 3 (10) |
| Thrombocytopenia | 3 (10) | 3 (10) |
| Acute kidney injury | 3 (10) | 1 (3) |
| Haemoptysis | 3 (10) | 0 |
| Leucocytosis | 3 (10) | 3 (10) |
| Depression | 3 (10) | 1 (3) |
| Dizziness | 3 (10) | 0 |
| Oral herpes | 3 (10) | 0 |

Treatment-emergent SAEs occurring in ≥5% of RP2D-treated participants were pneumonia (n=6; 21%), febrile neutropenia (n=5; 17%), sepsis (n=3; 10%), and infusion-related reactions (IRRs) (n=2; 7%). The most common (≥10%) treatment-related adverse events (TRAEs) (all grades) with the RP2D were IRRs (n=5; 17%) and febrile neutropenia (n=3; 10%). Grade ≥3 TRAEs occurring with the RP2D in ≥5% of participants were febrile neutropenia (n=3; 10%), IRRs (n=2; 7%), and anemia (n=2; 7%).

Three of the 68 participants experienced DLTs. Reversible VOD possibly related to pivekimab sunirine occurred in two participants (grade 3, 0.18 mg/kg after 2 cycles; grade 4, 0.45 mg/kg during cycle 1). Prolonged neutropenia (grade 3) considered related to pivekimab sunirine occurred in one participant (0.3 mg/kg after 2 cycles). TEAEs leading to treatment discontinuation occurred in seven of 68 participants (10%). The 30-day mortality rate was 7% in patients (2 deaths due to disease progression).

Thus, in sum, tolerable safety results were observed in R/R AML patients receiving 0.045 mg/kg pivekimab sunirine Q3W.

Anti-leukemia responses were assessed per International Working Group (IWG) criteria 2003 (Cheson B D, et al. Revised recommendations of the International Working Group for Diagnosis, Standardization of Response Criteria, Treatment Outcomes, and Reporting Standards for Therapeutic Trials in Acute Myeloid Leukemia. J Clin Oncol 2003; 21(24): 4642-9) and European LeukemiaNet (ELN) response criteria 2017 (Dohner H, et al. Diagnosis and management of AML in adults: 2017 ELN recommendations from an international expert panel. Blood 2017; 129(4): 424-47) as composite complete remission (CCR) rate (defined as CR without minimal residual disease [CRMRD−]+CR+CRh+CR with incomplete recovery CRi]). Overall response rate (ORR) was defined as CRMRD−+CR+CRh+CRi+morphologic leukemia-free state (MLFS) +partial response (PR). Duration of overall response (DOR) was also evaluated.

The response criteria for AML are shown in Table 6 below.

TABLE 6
Response criteria for AML.

| Response | Criteria |
|---|---|
| CR without minimal residual | CR with negativity for a genetic marker by reverse transcription |
| disease (CR_{MRD-}) | quantitative polymerase chain reaction (RT-qPCR), or CR with |
| | negativity by multiparameter flow cytometric (MFC) |
| Complete remission (CR) | a. Morphologic CR <5% blasts |
| | b. Absolute neutrophil count (ANC) >1000/μL (microliter) |
| | c. Platelets ≥100,000/μL (microliter) |
| | d. Patient independent of transfusions |
| | e. No residual evidence of active extramedullary disease |
| | f. MRD+ or unknown |
| Complete remission with | Meets requirements for CR except ANC >500/μL (microliter) |
| partial hematologic recovery | AND platelets >50,000/μL (microliter) |
| (CRh) | |
| Complete | Meets requirements for CR except either ANC <1000/μL |
| remission/response with | (microliter) or platelets <100,000/μL (microliter) |
| incomplete recovery (CRi) | |
| Morphologic leukemia-free | a. Bone marrow <5% blasts in an aspirate with spicules |
| state (MLFS) | b. No blasts with Auer rods or persistence of extramedullary |
| | disease |
| | c. Marrow should not merely be “aplastic”; at least 200 cells |
| | should be enumerated or cellularity should be at least 10% |
| Partial response (PR) | Decrease of at least 50% in the percentage of blasts to 5% to |
| | 25% in the BMA and the normalization of blood counts, as |
| | noted above |
| Relapse following complete | Reappearance of leukemia blasts in the peripheral blood or the |
| response | finding of more than 5% blasts in the bone marrow, not |
| | attributable to another cause (e.g., bone marrow regeneration |
| | after consolidation therapy [or extramedullary relapse]) |
| Stable disease (SD) | Absence of CR_{MRD-}, CR, CRh, CRi, PR, MLFS; and criteria for |
| | PD not met |
| Progressive disease (PD) | a. Evidence for an increase in bone marrow blast percentage |
| | and/or increase of absolute blast counts in the blood: |
| | i. Increased or persistent bone marrow disease without at least a |
| | 100% improvement (i.e., a doubling) in ANC to an absolute |
| | level (>0.5 × 10₉/L [500/μL (microliter)], and/or platelet count |
| | to >50 × 10₉/L [50,000/μL (microliter)] nontransfused): |
| | 1. >50% increase in bone marrow blasts over baseline |
| | (a minimum 15 percentage point increase is required in |
| | cases with <30% blasts at baseline); or |
| | 2. Persistent bone marrow blast percentage of >70% |
| | over at least 3 months |
| | ii. >50% increase in peripheral blasts (white blood cell count × |
| | % blasts) to >25 × 10₉/L (>25,000/μL (microliter)) (in the |
| | absence of differentiation syndrome); or |
| | iii. New extramedullary disease |

Response assessments were performed in bone marrow aspirates (BMAs) for differential assessments taken on Cycle 1, Day 21±7 days. Subsequent BMAs were performed approximately every 1 to 2 cycles as clinically indicated, and at the 30-Day Follow-up visit. Data was also collected in the event BMAs were performed more frequently. No repeat bone marrow was necessary if lack of response or PD could be unequivocally diagnosed from peripheral blood tests or if the bone marrow test was considered noncontributory at any time point.

At the RP2D of 0.045 mg/kg pivekimab sunirine Q3W, ORR was 21% (n= 6/29) and CCR rate was 17% (n= 5/29) (Table 7). ORR in participants with de novo (nonsecondary) AML was 33% (n= 6/18). Importantly, the ORR and CCR for venetoclax-naive, RP2D-treated participants were 27% (n= 4/15) and 20% (n= 3/15), respectively. ORR and CCR in those with prior venetoclax exposure for AML were lower, at 14% (n= 2/14) for both outcomes. At the RP2D, ORR and CCR in participants who had two prior lines were 27% (n= 4/15) and 20% (n= 3/15), respectively. Participants with prior HSCT achieved an ORR and CCR of 33% (n= 3/9) for both outcomes. At the RP2D, in participants with prior intensive therapy, both the ORR and CCR were 23% (n= 5/22). Prior intensive therapy was defined as anthracycline or high-dose cytarabine-based therapies, or chemotherapy-based conditioning for transplant. Individual responses were also seen in participants with FLT3-ITD (n=¼) and IDH1 (n=⅓) mutations.

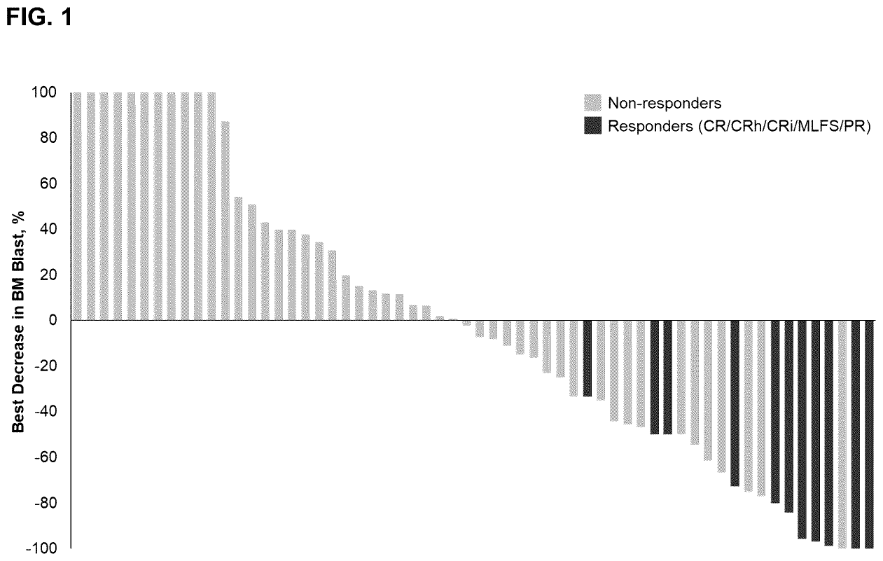

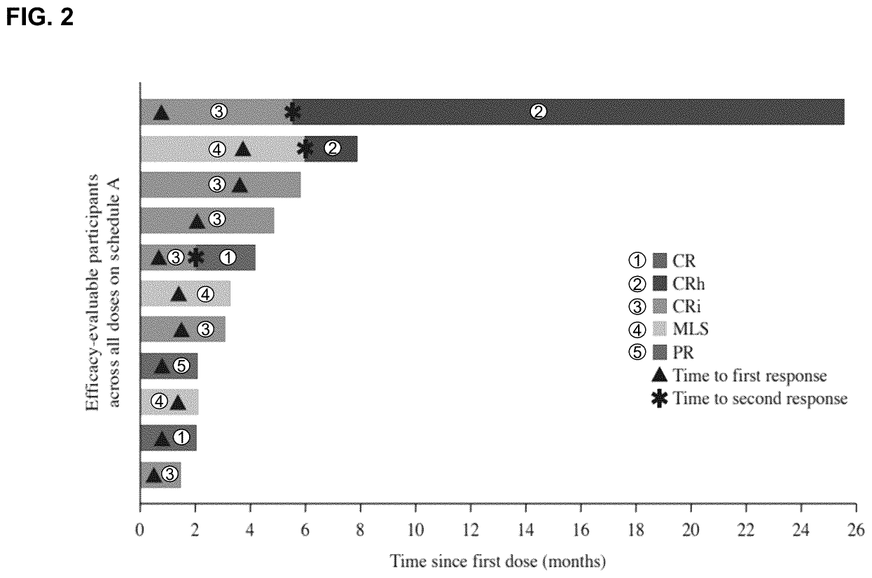

In order to enable time to response and duration of response estimates, responses were grouped across all dose levels in cohorts 1 and 2. Across all dose levels and cohorts, nearly half of participants (46% n= 31/68]) experienced decrease in bone marrow blasts, and ORR was 16% (n=68; 2 CR, 2 CRh, 4 CRi, 2 MLFS, 1 PR) (Table 7; FIG. 1). Median time to first CCR (CR/CRh/CRi) among all schedule A doses was 1.76 months (range, 0.46-4.37) (FIG. 2) and 2.04 months (range, 0.79-3.61) with the RP2D. Median duration of CCR (CR/CRh/CRi) among all schedule A doses was 2.22 months (95% CI, 1·02-3·55) and 2.27 months (95% CI, 1·64—not estimable) with the RP2D (Table 7).

TABLE 7
Response data in AML patients

| | All | | RP2D 0.045 | |
|---|---|---|---|---|
| | Participants | | mg/kg Q3W | |
| Response | (n = 68) | | (n = 29) | |
| ORR, No. (%), CR + CRh + CRi + | 11 | (16) | 6 | (21) |
| MLFS + PR | | | | |
| 95% CI | 8, 27 | | 8, 40 | |
| Best overall response, No. (%) | | | | |
| CR | 2 | (3) | 1 | (3) |
| CRh | 2 | (3) | 1 | (3) |
| CRi | 4 | (6) | 3 | (10) |
| MLFS | 2 | (3) | 1 | (3) |
| PR | 1 | (2) | 0 | |
| SD | 35 | (52) | 17 | (59) |
| PD | 16 | (24) | 5 | (17) |
| CCR rate, No. (%), CR + CRh + CRi | 8 | (12) | 5 | (17) |
| 95% CI | 5, 22 | | 6, 36 | |
| Median duration of remission (95% CI), | 2.0 | (1.0, 3.6) | 2.6 | (0.7, . . . ) |
| mo | | | | |
| Median duration of CR (95% CI), mo | 2.2 | (1.0, 3.6) | 2.3 | (1.6, . . . ) |

In this study in R/R AML patients, pivekimab sunirine monotherapy demonstrated anti-leukemia activity and tolerable safety in heavily pretreated patients with poor risk characteristics, leading to selection of the RP2D at 0.045 mg/kg for treatment.

pivekimab sunirine, with its novel single-strand DNA alkylating payload, demonstrated tolerability across a wide range of doses, with no maximum tolerated dose (MTD) defined over a 30-fold dose escalation (0.015-0.45 mg/kg Q3W). Overall, the frequency and grade of hematologic TEAEs were similar to other AML therapies, likely reflecting the effect of underlying disease as opposed to drug-related AEs. For example, the incidence of febrile neutropenia (35%) in all schedule A participants was comparable to that published for ivosidenib (29%) and gilteritinib (46%). pivekimab sunirine treatment was associated with IRRs (31%)—mostly tachycardia and/or chills occurring with the first dose—which were primarily grade 1-2 in severity. Only one grade 3 case led to discontinuation; no grade 4 IRRs occurred. After the protocol was amended to include an additional steroid dose on the day before pivekimab sunirine infusion, IRR frequency decreased. Reversible VOD was seen in 3% (N= 3/91) of participants at dose levels four- to ten-fold higher per cycle than the selected RP2D.

Despite a high proportion of baseline adverse-risk characteristics, anti-leukemia responses were observed across all but one dose level (0.45 mg/kg) in dose-escalation cohorts and both dose-expansion cohorts. At the RP2D (0.045 mg/kg Q3W), ORRs were seen across multiple high-risk subgroups, including participants with prior venetoclax (14%), prior intensive therapy (23%), and prior HSCT (33%). In RP2D-treated participants, most (66%) were treated with two to three prior therapies. The ORR was 27% (n= 4/15) in less heavily pretreated participants (i.e., those with only two prior lines of AML therapy).

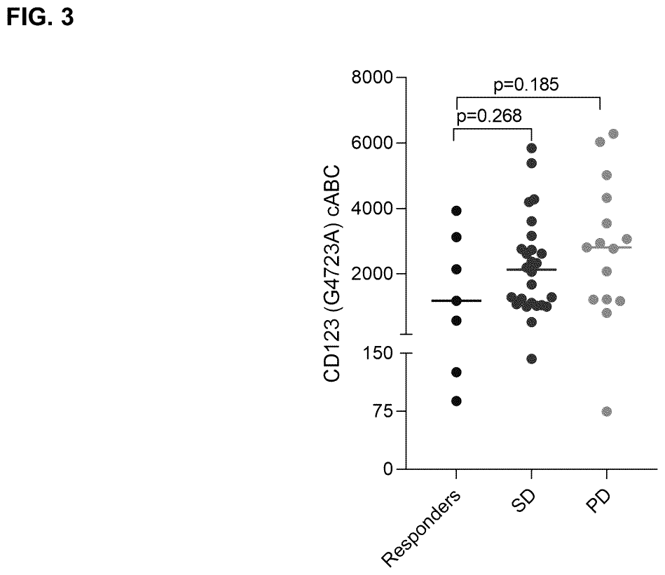

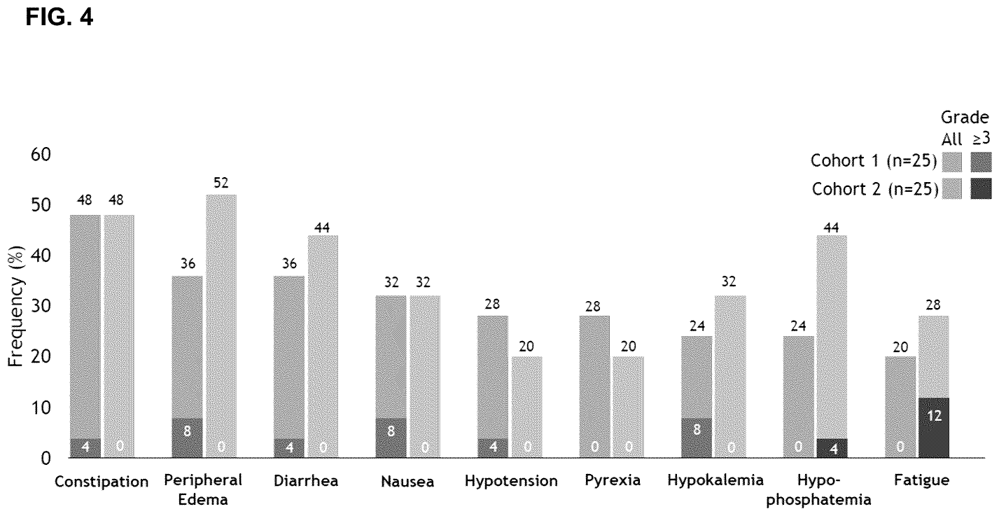

Unlike mutation-specific treatment options, pivekimab sunirine is not restricted to specific subpopulations, and anti-leukemia activity was observed in several high-risk subgroups. Surprisingly, pivekimab sunirine had anti-leukemia activity regardless of CD123 expression, demonstrating that patients do not need to be selected for treatment based on level of CD123 expression (FIG. 3).

In this study, pivekimab sunirine at the RP2D demonstrated anti-leukemia activity, with a CCR rate of 17% and a tolerable safety profile in R/R AML.

### Example 2: Treatment of AML in Human Patients Using Pivekimab Sunirine in Combination with AZA and VEN

### Part 1)

An open-label, multicenter, Phase 1b/2 study of pivekimab sunirine in combination with azacitidine (AZA) and venetoclax (VEN) was performed in adults with newly diagnosed (ND) CD123-positive AML (any CD123+ expression by local flow cytometry or IHC). Patients received the recommended phase 2 dose of pivekimab sunirine 0.045 mg/kg IV on Day 7 (D7)+AZA 75 mg/m² SC or IV daily on D1-7 in a 28-day cycle. Patients in Cohort 1 also received VEN up to 400 mg orally (PO) daily for at least 14 days, and patients in Cohort 2 received VEN up to 400 mg PO daily for up to 28 days (based on cohort assignment) in the 28-day cycle. Bone marrow assessments were performed on Days 12-14 (cohort 1) or Days 18-21 (cohort 2) of cycle 1 to determine VEN duration. The primary endpoints were composite CR rate (CCR [CR+CRh+CRp (Meets requirements for CR except platelets <100,000/μL (microliter))+CRi]), MRD rate (assessed centrally [Hematologics, Inc.] by flow cytometry; <0.1% defined as negative) and duration of remission. Safety was also evaluated. Responses were determined using ELN 2017 criteria (with the addition of CRh) and a 14-day post-marrow count recovery window.

Following preparation, IMGN632 (or pivekimab sunirine (pvek)) was administered by IV syringe pump. Overall length of infusion was varied depending on dose and patient tolerability. Initially, the study drug was administered at a rate of 0.165 mg/min (0.8 m/min); and, after 30 minutes, if well tolerated, the rate was increased to 0.33 mg/min. Subsequent infusions were delivered at the highest tolerated rate. Before and following infusion, the line was flushed with 5% dextrose to ensure delivery of the full dose. Patients were carefully observed during each infusion and vital signs were monitored. Patients remained in the clinic under observation for 4 hours after the first infusion and for at least 1 hour after each subsequent infusion.

Baseline characteristics of patients in Cohort 1, Cohort 2, and the overall patient population are provided in Table 8 below.

TABLE 8
Baseline Characteristics

| | | Cohort 1 | | Cohort 2 | | | |
|---|---|---|---|---|---|---|---|
| | | PVEK + AZA + V | | PVEK + AZA + V | | Overall | |
| | | EN ≥14 days | | EN ≤28 days | | Population | |
| | | (n = 25) | | (n = 25) | | (N = 50) | |
| Age | Median(Range) | 74 | (46-83) | 74 | (59-83) | 74 | (46-83) |
| | ≥75 y, % (n) | 36% | (9) | 48% | (12) | 42% | (21) |
| Sex, % (n) | Male | 64% | (16) | 64% | (16) | 64% | (32) |
| | Female | 36% | (9) | 36% | (9) | 36% | (18) |
| ECOG PS, % | 0 | 40% | (10) | 36% | (9) | 38% | (19) |
| (n) | 1 | 60% | (15) | 64% | (16) | 62% | (31) |
| AML | De novo | 80% | (20) | 76% | (19) | 78% | (39) |
| classification, | Secondary | 20% | (5) | 24% | (6) | 22% | (11) |
| % (n) | | | | | | | |
| ELN 2017 | Favorable | 4% | (1) | 4% | (1) | 4% | (2) |
| risk,% (n) | Intermediate | 36% | (9) | 16% | (4) | 26% | (13) |
| | Adverse | 60% | (15) | 72% | (18) | 66% | (33) |
| | Missing/not | 0% | (0) | 8% | (2) | 4% | (2) |
| | determined | | | | | | |
| Selected | TP53 | 20% | (4/20) | 53% | (10/19) | 36% | (14/39) |
| mutations, % | IDH1/2 | 32% | (7/22) | 9% | (2/22) | 21% | (9/44) |
| (n/N)^{a,b} | NPM1 | 17% | (4/23) | 19% | (4/21) | 18% | (8/44) |
| | K/NRAS | 19% | (4/21) | 11% | (2/19) | 15% | (6/40) |
| | FLT3- | 17% | (4/23) | 9% | (2/22) | 13% | (6/45) |
| | TKD/ITD | | | | | | |

^{a}Characteristics in bold represent notable differences in incidence of mutations between cohorts
^{b}There are patients without complete molecular data available

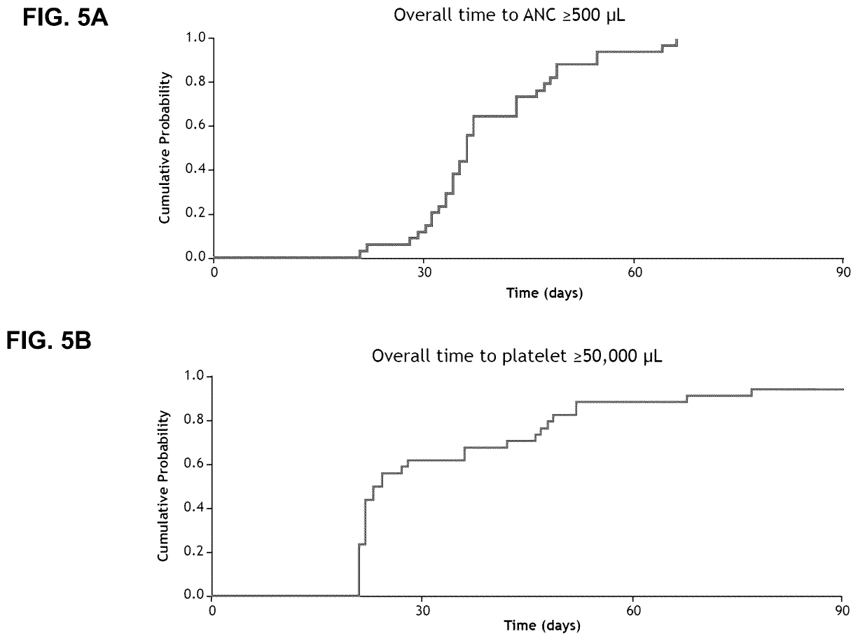

The most common non-hematologic treatment-emergent adverse events (TEAE) (all grades [grade 3+]) seen in >20% of all patients are shown in FIG. 5. Two patients (4%) discontinued treatment due to adverse events (generalized edema and prolonged myelosuppression/marrow hypoplasia). The 30-day mortality rate was 0%, and the 60-day mortality was 4%. Selected TEAEs in the overall population are shown in Table 9 below.

TABLE 9
Selected TEAEs in the Overall Population

| | All Grades | Grade 3 |
|---|---|---|
| Edema events^{a} | | |
| Peripheral edema | 44% | 4% |
| Generalized edema | 6% | 4% |
| Infusion-related reactions (IRRs)^{b} | 16% | 0% |
| Hepatotoxicity | | |
| ALT/AST elevation | 8% | 4% |
| Hyperbilirubinemia | 2% | 0% |
| VOD/SOS | 0% | 0% |

^{a}Preferred terms (MedDRA v24.0) under edema include peripheral edema, generalized edema, fluid overload, peripheral swelling, localized edema, fluid retention
^{b}To mitigate IRRs, the prophylaxis regimen includes additional steroid doses on the day before the pivekimab sunirine infusion

Forty-eight (48) percent of patients had at least one edema adverse event, which were mostly grade 1-2, with no grade 4 events. The median time to onset for an edema event (all grades) was 23 days (range 1-243). Seventy-four (74) percent of all edema events resolved, and the median time to resolution for all-grade edema events was 10 days (range 1-87). Thirty-seven (37) percent of edema events were treated with diuretic(s) for a median of 7.5 days (range 1-478); seventeen (17) percent of events used at least two diuretics. No capillary leak syndrome (CLS) events were reported. Concurrent albumin levels <3 g/dL occurred in only 15% of edema events.

Cycle delays, summarized in Table 10 below, were manageable and consistent with the median post-remission cycle delay of 13 days as in the AZA+VEN arm as published in the VIALE-A trial. (Pratz K W, et al. Am J Hematol. 2022; 97(11):E416 E416-E419.)

TABLE 10
Cycle Delays

| | Blast Clearance | Post-Remission |
|---|---|---|
| | Cycle^{b,c} | Cycles^{b} |
| Median, days (range)^{a} | 11 (1-51) | 13 (−2-63) |

^{a}From day 28 of each cycle
^{b}In patients achieving CCR
^{c}Reported by first cycle achieving blast clearance

The count recovery kinetics are shown in FIGS. 5A and 5B and in Table 11 below. Twenty-six (26) percent of patients had concomitant G-CSF use.

TABLE 11
Count Recovery Kinetics^{a}

| | ANC ≥500/μl | PLT ≥50k/μl |
|---|---|---|
| | Median, days (range) | Median, days (range) |
| Blast clearance cycle | 34 (20-55) | 22 (20-52) |
| Post-remission cycles | 28 (20-132) | 22 (20-132) |

^{a}In patients achieving CCR

By cycle 3, ninety-seven (97) percent of patients on treatment had <14 days of VEN per cycle. Twenty-eight (28) percent of patients had AZA dose modifications.

The median time to MRD clearance was 1.87 months (range, 0.79-5.16). Response rates and MRD negativity were comparable between cohorts 1 and 2. In patients with a duration of VEN ≤14 days (n=21) in cycle 1, 76% of patients had a best overall response of CCR. In patients with a duration of VEN ≥22 days (n=20) in cycle 1, 75% of patients had a best overall response of CCR. Similar CCR rates were observed, despite the difference in VEN duration

Response rates are shown in Table 12, and responses in particular subsets of interest are shown in Table 13. The treatment shows broad anti-leukemia activity across molecular subsets. There were no substantial differences in responses between cohorts 1 and 2 in the subset analyses. The study population was enriched for the adverse molecular subset of TP53^{mut} (36%).

TABLE 12
Anti-leukemia Activity^{a}

| | CR rate | CCR rate^{b} | CCR_{mrd-}^{c} |
|---|---|---|---|
| Overall Population | 54% (27/50) | 68% (34/50) | 76% (22/29) |
| (N = 50) | | | |
| Meets unfit FDA | 61% (14/23) | 78% (18/23) | 79% (11/14) |
| criteria (n = 23) | | | |

^{a}Responses determined by ELN 2017 criteria (with addition of CRh)
^{b}CCR = CR + CRh + CRp + CRi
^{c}MRD rate (assessed centrally [HematoLogics, Inc.] by flow cytometry; <0.1% defined as negative)
^{d}Unfit by FDA criteria includes patients ≥75 years old, or younger patients with defined organ dysfunction; the 27 patients who were not included in unfit population by FDA criteria were <75 years old without defined comorbidities

TABLE 13
Anti-leukemia activity in subsets of interest

| | | | Pivekimab Sunirine Triplet |
|---|---|---|---|
| TP53 status | Wildtype | CCR | 88% (22/25) |
| | | CR | 84% (21/25) |
| | | MRD− | 80% (16/20) |
| | Mutant | CCR | 50% (7/14) |
| | | CR | 21% (3/14) |
| | | MRD− | 50% (3/6) |
| FLT3 ITD or TKD | | CCR | 100%(6/6) |
| | | MRD− | 100%(6/6) |
| IDH1^{mut} | | CCR | 100% (4/4) |
| | | MRD− | 67% (2/3) |
| IDH2^{mut} | | CCR | 100% (6/6) |
| | | MRD− | 83% (5/6) |
| NPM1^{mut} | | CCR | 100%(8/8) |
| | | MRD− | 86%(6/7) |
| K/NRAS^{mut} | | CCR | 50% (3/6) |
| | | MRD− | 67% (2/3) |

CCR = CR + CRh + CRp + CRi
MRD rate assessed by flow cytometry; <0.1% defined as negative

A pooled analysis of the phase 3 VIALE-A trial and a phase 1b trial demonstrated that risk stratification based on molecular features predicted response better than ELN/cytogenetic risk. (Dohner H, et al. Presented at: 2022 American Society of Hematology Annual Meeting. Dec. 11, 2022. New Orleans, LA. Abstract 602.) The higher benefit group was found to be TP53^{wt}, no FLT3-ITD, and K/NRAS^{wt}. The intermediate benefit group was TP53^{wt} and FLT3-ITD or K/NRAS^{mut}. The lower benefit group was TP53^{mut}. The effects of treating patients in these molecular risk categories with pivekimab sunirine, AZA, and VEN (pivekimab sunirine triplet) are shown in Table 14.

TABLE 14
Molecular stratifications in subsets of interest

| | | Pivekimab Sunirine |
|---|---|---|
| | | Triplet |
| Higher benefit | CCR | 94% (17/18) |
| | CR | 89% (16/18) |
| | MRD− | 73% (11/15) |
| Intermediate benefit | CCR | 71% (5/7) |
| | CR | 71% (5/7) |
| | MRD− | 100% (5/5) |
| Lower benefit | CCR | 50% (7/14) |
| | CR | 21% (3/14) |
| | MRD− | 50% (3/6) |

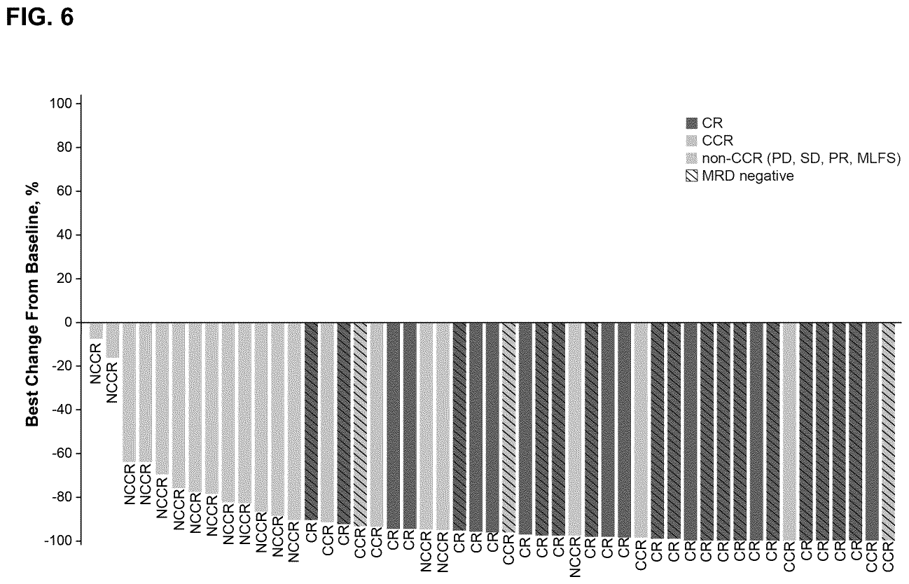

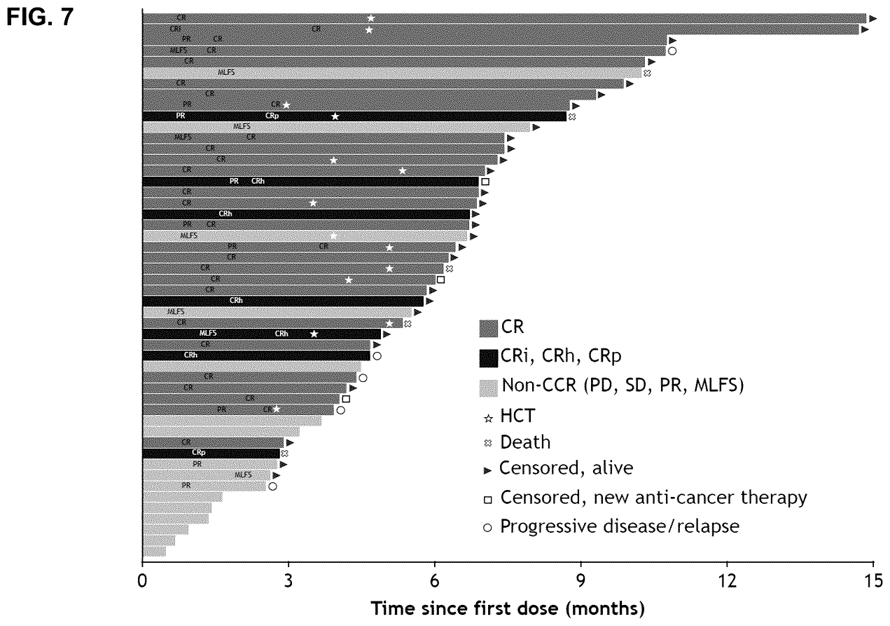

Best overall responses are shown in FIG. 6. In the overall population, the CCR MRD-negative rate was 76% ( 22/29). Of the MRD-negative patients, all except one had undetectable disease below the lower limit of detection (0.02%.) The duration of overall response is shown in FIG. 7. 14 patients (28% of overall population; 41% of those achieving CCR) underwent consolidative HCT after completing study treatment. The median duration of follow up was 5.16 months.

Landmark overall survival estimates in the overall population are shown in Table 15.

TABLE 15
Landmark overall survival estimates

| | Triplet Estimate |
|---|---|
| Landmark, mos | (95% CI) |
| 3 | 92% (80-97) |
| 6 | 86% (72-94) |

Overall, the regimen was well tolerated with no new safety signals, and the addition of pivekimab sunirine to the AZA-VEN backbone did not appear to meaningfully prolong count recovery (with ANC ≥500/μL and platelet ≥50 k/μL recovery times of 34 and 22 days, respectively). The triplet regimen demonstrated similar post-remission cycle delays (13 days), as what has been published in the VEN-AZA doublet of the VIALE-A trial. With a manageable safety profile in patients with newly diagnosed AML, the pivekimab sunirine+AZA+VEN triplet demonstrated anti-leukemia activity with high rates of CR, CCR, and MRD negativity. The robust CR rates and early MRD negative responses in a cohort enriched for adverse disease characteristics are particularly surprising. In addition, broad anti-leukemia activity was observed across molecular subsets, and the time to achieving MRD clearance indicates rapid and deep disease control.

### Part 2)

The following summarizes data and results obtained from the Phase 1b dose escalation (0.015 mg/kg) phase of an open-label, multicenter, Phase 1b/2 study of pivekimab sunirine (PVEK) when administered as combination and monotherapy regimens. The multicenter study includes Regimen A (PVEK+azacitidine [AZA]), Regimen B (PVEK+venetoclax [VEN]), Regimen C (PVEK+VEN+AZA), and Regimen D as monotherapy.

This portion of the study was, in part, to determine the recommended phase 2 dose (RP2D) of PVEK in Regimen C and further characterize the safety profile and assess antileukemia activity of Regimen C in AML patient populations.

A total of 49 subjects with untreated AML and preexisting comorbidities that preclude use of intensive chemotherapy were enrolled in Regimen C. The primary objective was assessing the rate of complete remission (CR). CR was defined using criteria in the Viale-A protocol. (See, DiNardo et al. (2020) Azacitidine and venetoclax in previously untreated acute myeloid leukemia. The New England Journal of Medicine. 383(7):617-629.)

### Demographic, Baseline Characteristics and Exposure Summary.

Key demographic and baseline characteristics are listed in Table 1. Thirty-six (73.5%) subjects were 75 years or older. A total of 17 (34.7%) had TP53 mutation.

TABLE 1
Key Demographic and Baseline Characteristics

| | Frontline Unfit | |
|---|---|---|
| | AML Subjects | |
| | (N = 49) | |
| Age - Median (Range) | 77 | (58, 85) |
| Age Group - n (%) | | |
| <75 | 13 | (26.5) |
| ≥75 | 36 | (73.5) |
| ECOG Performance Status - n (%) | | |
| 0 | 14 | (28.6) |
| 1 | 28 | (57.1) |
| 2 | 6 | (12.2) |
| 3 | 1 | (2.0) |
| Cytogenetics - n (%) | 9 | (18.4) |
| Favorable | 28 | (57.1) |
| Intermediate | 6 | (12.2) |
| Adverse | 1 | (2.0) |
| Not Determined Unknown | 4 | (8.2) |
| Bone Marrow | | |
| Blast Count - n (%) | | |
| ≥20% | 46 | (93.9) |
| <30% | 22 | (44.9) |
| ≥30% to <50% | 10 | (20.4) |
| ≥50% | 16 | (32.7) |
| Type of AML - n (%) | | |
| De Novo | 42 | (85.7) |
| Secondary | 7 | (14.3) |
| FLT3 Mutation - n (%) | 10 | (20.4) |
| IDH1 Mutation - n (%) | 1 | (2.0) |
| IDH2 Mutation - n (%) | 3 | (6.1) |
| TP53 Mutation - n (%) | 17 | (34.7) |
| NPM1 Mutation - n (%) | 11 | (22.4) |

Median durations of PVEK, VEN and AZA were 5.5 months (range: 0.7, 22.7), 5.1 months (range: 0.9, 22.9) and 5.7 months (range: 0.9, 23.4), respectively. The median number of cycles of PVEK, VEN and AZA were 4 (range: 1, 20), 4 (range: 1, 20) and 4 (range: 1, 21), respectively. Median relative dose intensities of PVEK, VEN, and AZA were 62.9% (range: 35%, 100%), 42.2% (range: 14%, 93%) and 80% (range: 43%, 100%), respectively.

Of 49 subjects, 10 subjects were assigned to receive venetoclax for at least 14 days in each cycle and 39 subjects were assigned to receive venetoclax for 28 days or less in each cycle. Of the 10 subjects with a target venetoclax dosing of at least 14 days, median durations of PVEK and VEN were 4.3 months (range: 1.9, 22.7) and 4.5 months (range: 2.1, 22.9), respectively. Of the 39 subjects with a target venetoclax dosing of 28 days or less, median durations of PVEK and VEN were 5.8 months (range: 0.7, 13.0) and 5.7 months (range: 0.9, 15.2), respectively.

### Efficacy

At time of this assessment, the median follow-up of the 49 frontline unfit AML subjects was 10.0 months (range: 1.2+, 26.7). The efficacy results are summarized in Table 2A, Table 2B and Table 2C. Overall Survival (OS) data was 10.28 months, though this data was not fully matured at time of this assessment. As such, median OS may change with longer follow-up.

The proportion of TP53-mutated subjects (34.7%) was higher than anticipated and those subjects are known to have poor prognosis. Therefore, if the proportion of TP53-mutated subjects were lower, a higher efficacy outcome would be expected. See, Table 2B. For example, in a population wherein the proportion of TP53-mutated subjects was equal to 23%, as observed on the VEN+AZA arm in Viale-A (Id.), the predicted CR rate would be 69.4% (95% PI: 57.1, 79.6), the predicted median OS would be 19.4 months (95% PI: 9.7, −), and the predicted 6-, 9-, and 12-month survival rate would be 85.7%, 76.7%, and 56.3%, respectively. These predicted values would reflect the predicted efficacy outcome in these 49 subjects, if the proportion of TP53-mutated subjects was 23% while all other population characteristics remained the same.

TABLE 2A
Key Efficacy Results

| | PVEK + VEN + AZA | |
|---|---|---|
| | (N = 49) | |
| CR Rate | | |
| n (%) | 31 | (63.3) |
| (95% CI) | (48.3, 76.6) | |
| Overall Survival | | |
| Median (95% CI) | 10.3 months | (9.1, 21.5) |
| 6-month rate (%) | 84 | (70, 91) |
| 9-month rate (%) | 71 | (55, 82) |
| 12-month rate (%) | 46 | (28, 63) |

TABLE 2B
Key Efficacy Results by TP53 Mutation Status

| | TP53 Mutated | TP53 Not Detected |
|---|---|---|
| | (N = 17) | (N = 32) |
| CR Rate | | |
| n (%) | 6 (35.3) | 25 (78.1) |
| 95% | 14.2, 61.7 | 60.0, 90.7 |
| CI | | |
| DoCR | | |
| Median (95% CI) | 8.0 months (4.1, —) | 16.1 months (7.5, —) |
| 6-month rate (%) | 67 (19, 90) | 84 (62, 94) |
| 9-month rate (%) | 0 | 66 (36, 84) |
| 12-month rate (%) | 0 | 55 (24, 78) |
| CR MRD− Rate | | |
| n (%) | 6 (35.3) | 17 (53.1) |
| 95% | 14.2, 61.7 | 34.7, 70.9 |
| CI | | |
| Overall Survival | | |
| Median OS (95% CI) | 8.5 months (4.7, 10.1) | 21.5 months (12.4, —) |
| 6-month rate (%) | 71 (43, 87) | 91 (73, 97) |
| 9-month rate (%) | 44 (20, 66) | 87 (70, 95) |
| 12-month rate (%) | 0 | 75 (51, 89) |

The median duration of CR was 9.4 months (95% CI: 7.5, −); 48% of subjects remained in remission at 12 months. Twenty-three (46.9%) subjects achieved both CR and MRD <10⁻³. Among the 49 subjects, 8 (16.3%) received stem cell transplant (SCT) post-study treatment. SAFETY

### Adverse Events.

At the time of this assessment, the median duration of PVEK among 49 patients was 5.5 months (range: 0.7, 22.7).

Treatment-emergent AEs were observed in all 49 (100%) of subjects. Grade ≥3 AEs were observed in 48 (98%) subjects. The most common AEs (≥15%) and grade ≥3 AEs (≥5%) are listed in Table 3.

TABLE 3
Treatment-Emergent Adverse Events

PVEK + VEN + AZA
(N = 49)

| Adverse events - n (%) | Any grade | Grade ≥ 3 |
|---|---|---|
| All adverse events | 49 (100) | 48 (98.0) |
| Neutropenia* | 34 (69.4) | 31 (63.3) |
| Thrombocytopenia* | 34 (69.4) | 32 (65.3) |
| Constipation | 30 (61.2) | 1 (2.0) |
| Oedema peripheral | 25 (51.0) | 2 (4.1) |
| Nausea | 24 (49.0) | 0 |
| White blood cell count | 24 (49.0) | 24 (49.0) |
| decreased | | |
| Anaemia* | 22 (44.9) | 18 (36.7) |
| Diarrhoea | 20 (40.8) | 2 (4.1) |
| Hypophosphataemia | 20 (40.8) | 3 (6.1) |
| Hypokalaemia | 19 (38.8) | 5 (10.2) |
| Febrile neutropenia | 17 (34.7) | 17 (34.7) |
| Hypoalbuminaemia | 15 (30.6) | 1 (2.0) |
| Infusion related reaction | 15 (30.6) | 1 (2.0) |
| Dizziness | 14 (28.6) | 1 (2.0) |
| Fatigue | 13 (26.5) | 3 (6.1) |
| Decreased appetite | 13 (26.5) | 1 (2.0) |
| Hypertension | 13 (26.5) | 7 (14.3) |
| Cough | 11 (22.4) | 1 (2.0) |
| Pain in extremity | 11 (22.4) | 0 |
| Hypotension | 10 (20.4) | 2 (4.1) |
| Pyrexia | 10 (20.4) | 0 |
| Dysgeusia | 9 (18.4) | 1 (2.0) |
| Pneumonia | 9 (18.4) | 8 (16.3) |
| Dyspnoea | 8 (16.3) | 1 (2.0) |
| Hyperglycaemia | 8 (16.3) | 1 (2.0) |
| Hyponatraemia | 8 (16.3) | 0 |
| Insomnia | 8 (16.3) | 0 |
| Pleural effusion | 8 (16.3) | 2 (4.1) |
| Vomiting | 8 (16.3) | 1 (2.0) |
| Sepsis | 6 (12.2) | 6 (12.2) |
| Lymphocyte count decreased | 5 (10.2) | 5 (10.2) |
| Bone marrow failure | 3 (6.1) | 3 (6.1) |

*Neutropenia = group PTs of neutropenia and neutrophil count decreased; Thrombocytopenia = group PTs of thrombocytopenia and platelet count decreased; Anaemia = grouped PTs of anaemia and hemoglobin decreased.

SAEs were observed in 38 (77.6%) subjects. The most common SAEs (≥5%) were febrile neutropenia (n=11; 22.4%), pneumonia (n=6; 12.2%), and sepsis (n=6; 12.2%).

AEs leading to PVEK discontinuation occurred in 3 (6.1%) subjects. Fatal AEs occurred in 2 (4.1%) subjects with multiple organ dysfunction syndrome and left ventricular dysfunction.

### Additional Data

Efficacy and safety of PVEK+VEN+AZA (IMGN632-0802) and VEN+AZA (VIALE-A) are summarized below. Only publicly available data for VEN+AZA from VIALE-A is included.

TABLE 4
Demo and Disease Characteristics.

| | PVEK + VEN + | VIALE-A ¹ |
|---|---|---|
| | AZA | VEN + AZA |
| | (N = 49) | (N = 286) |
| Age | | |
| Median (range) -yr | 77 (58-85) | 76 (49-91) |
| ≥75 yr - n (%) | 36 (73.5) | 174 (61) |
| AML type - n (%) | | |
| De Novo | 42 (85.7) | 214 (75) |
| Secondary | 7 (14.3) | 72 (25) |
| Somatic Mutation - n (%) | | |
| IDH1 or IDH2 | 4/41 (9.8) | 61/245 (25) |
| FLT3 | 10/44 (22.7) | 29/206 (14) |
| NPM1 | 11/43 (25.6) | 27/163 (17) |
| TP53 | 17/49 (34.7) | 38/163 (23) |

TABLE 5A
CR, Duration of CR, and OS.

| | | PVEK + VEN + | VIALE-A ** |
|---|---|---|---|
| | PVEK + VEN + AZA | AZA | VEN + |
| | Observed * | Predicted \hat{ } | AZA |
| | (N = 49) | (N = 49) | (N = 286) |
| CR Rate | | | |
| (%) | 63.3 | 69.4 | 38.8% |
| (95% CI or PI) | (48.3, 76.6) | (57.1, 79.6) | |
| Median Follow-up | 10.0 months | — | 43.2 months |
| Median Duration of CR | 9.4 months | — | 22.1 months |
| Overall Survival | | | |
| Median (95% CI or PI) | 10.3 months (9.1, 21.5) | 19.4 months (9.7, —) | 14.7 months |
| 6-month rate (%) | 84 (70, 91) | 85.7 (75.5, 95.8) | 73% |
| 9-month rate (%) | 71 (55, 82) | 76.7 (65.0, 87.6) | 64% |
| 12-month rate (%) | 46 (28, 63) | 56.3 (40.8, 71.2) | 57% |

* Observed efficacy data from the 49 subjects.
\hat{ } Predicted efficacy in a hypothetical population of 49 subjects where 23% subjects have TP53 mutation, assuming all other population characteristics remain the same. Prediction does not adjust any other potential differences in patient population from Viale-A. Comparison of the predicted outcome with Viale-A should be done with caution; analysis based on the published summary statistics from Viale-A.
** Pratz et al. (2024) Long-term follow-up of VIALE-A: Venetoclax and azacitidine in chemotherapy-ineligible untreated acute myeloid leukemia. American Journal of Hematology. 99(4): 615-624.

TABLE 5B
Overall Survival by TP53 Mutation Status.

| | | | VEN + AZA^{a,b} | |
|---|---|---|---|---|
| | | | | Intermediate |
| | PVEK + VEN + AZA | | Poor | Cytogenetics |
| | With | Without | Cytogenetics | without |
| | TP53 | TP53 | with TP53 | TP53 |
| | (N = 17) | (N = 32) | (N = 54) | (N = 166) |
| Median OS | 8.5 months | 21.5 months | 5.17 months | 19.2 months |

^{a}Pooled data.
^{b}Pollyea et al. (2022) Outcomes in patients with poor cytogenetics with or without TP53 mutations treated with venetoclax and azacitidine. Clinical Cancer Research. 28(24): 5272-5279.

TABLE 6
Safety

| | PVEK + VEN + | | VEN + | |
|---|---|---|---|---|
| | AZA | | AZA² | |
| | (N = 49) | | (N = 283) | |
| | Any | | Any | |
| Adverse events - n (%) | grade | Grade ≥ 3 | grade | Grade ≥ 3 |
| All adverse events | 49 (100) | 48 (98.0) | 283 (100) | 279 (99) |
| Hematologic adverse events | | | | |
| Thrombocytopenia* | 34 (69.4) | 32 (65.3) | 134 (47) | 130 (46) |
| Febrile neutropenia | 17 (34.7) | 17 (34.7) | 121 (43) | 121 (43) |
| Neutropenia* | 34 (69.4) | 31 (63.3) | 121 (43) | 121 (43) |
| Anemia* | 22 (44.9) | 18 (36.7) | 87 (31) | 80 (28) |
| Non-hematologic adverse events | | | | |
| Diarrhea | 20 (40.8) | 2 (4.1) | 128 (45) | 13 (5) |
| Nausea | 24 (49.0) | 0 | 126 (45) | 5 (2) |
| Constipation | 30 (61.2) | 1 (2.0) | 124 (44) | 4 (1) |
| Hypokalemia | 19 (38.8) | 5 (10.2) | 84 (30) | 33 (12) |
| Vomiting | 8 (16.3) | 1 (2.0) | 85 (30) | 6 (2) |
| Decreased appetite | 13 (26.5) | 1 (2.0) | 79 (28) | 14 (5) |
| Pyrexia | 10 (20.4) | 0 | 75 (27) | 6 (2) |
| Peripheral edema | 25 (51.0) | 2 (4.1) | 70 (25) | 1 (<1) |
| Fatigue | 13 (26.5) | 3 (6.1) | 62 (22) | 10 (4) |
| Hypophosphatemia | 20 (40.8) | 3 (6.1) | 17 (12) | 11 (8) |
| Hypertension | 13 (26.5) | 7 (14.3) | 30 (11) | 21 (7) |
| Infections | | | | |
| Pneumonia | 9 (18.4) | 8 (16.3) | 74 (26) | 66 (23) |
| Sepsis | 6 (12.2) | 6 (12.2) | 22 (8) | 21 (7) |

*For PVEK + VEN + AZA: Neutropenia = group PTs of neutropenia and neutrophil count decreased; Thrombocytopenia = group PTs of thrombocytopenia and platelet count decreased; Anaemia = grouped PTs of anaemia and hemoglobin decreased.

### Summary of Data and Results in Study of 49 Newly Diagnosed Chemo-Ineligible AML Subjects Who Received PVEK+VEN+AZA

Efficacy: CR rate was 63.3% (95% CI: 48.3, 76.6). Twenty-three (46.9%) subjects achieved both CR and measurable residual disease response (MRD <10-3). With a median follow-up of 10.0 months (range: 1.2+, 26.7), the median duration of CR was 9.4 months (95% CI: 7.5, −); 48% of subjects who achieved CR remained in remission at 12 months. Median OS was 10.3 months (95% CI: 9.1, 21.5). The 6-, 9-, and 12-months survival rates were 84%, 71%, and 46%, respectively.

Of the 49 subjects, 17 (34.7%) had TP53 mutation, which was higher than anticipated. However, should the proportion of TP53-mutated subjects be lower, a better efficacy outcome than that currently observed would be expected.

In sum, PVEK in combination with VEN and AZA demonstrated a high CR rate. With 10 months median of follow-up at time of analysis, the observed median OS is 10.3 months (95% CI: 9.1, 21.5). The projected median OS when adjusting baseline TP53 mutation status to reflect VIALE-A is 19.4 months (95% PI: 9.7, −).

Safety: PVEK in combination with VEN and AZA is well tolerated in frontline unfit AML subjects and the overall safety profile was manageable. There were no new safety indicators observed other than what is known to VEN+AZA in comparable patient population.

It is to be appreciated that the Detailed Description section, and not the Summary and Abstract sections, is intended to be used to interpret the claims. The Summary and Abstract sections sets forth one or more, but not all, exemplary aspects of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

What is claimed is:

1. A method of treating an unfit newly diagnosed acute myeloid leukemia (ND AML) in a subject comprising administering to the subject a combination therapy comprising (i) pivekimab sunirine, (ii) a BCL-2 inhibitor, and (iii) a hypomethylating agent.

2. A method for treating an acute myeloid leukemia (AML) in a subject comprising administering to the subject a combination therapy comprising (i) pivekimab sunirine, (ii) a BCL-2 inhibitor, and (iii) a hypomethylating agent, wherein the administration achieves a complete response (CR), CR (clinical) with partial hematological recovery (CRh), CR with incomplete platelet recovery (CRp), or CR (clinical) with incomplete recovery (CRi) with a duration of at least 3 months.

3. A method for treating a FLT3 TKD, IDH1^{mut}, NMP1^{mut}, and/or K/NAS^{mut} acute myeloid leukemia (AML) in a subject with comprising administering to the subject a combination therapy comprising (i) pivekimab sunirine, (ii) a BCL-2 inhibitor, and (iii) a hypomethylating agent.

4. A method of clearing minimal residual disease (MRD) in a human subject with acute myeloid leukemia (AML) comprising administering to the subject a combination therapy comprising (i) pivekimab sunirine, (ii) a BCL-2 inhibitor, and (iii) a hypomethylating agent, wherein the MRD is cleared in 2 months or less.

5. The method of any one of claims 1, 3, and 4, wherein the administration of the combination therapy achieves a complete response with a duration of at least 3 months.

6. The method of claim 2 or 5, wherein the duration of the CR, CRh, CRp, or CRi is at least 4 months.

7. The method of claim 2 or 5, wherein the duration of the CR, CRh, CRp, or CRi is at least 5 months or at least 6 months.

8. The method of claim 2 or 5, wherein the duration of the CR, CRh, CRp, or CRi is at least 10 months or at least 12 months.

9. The method of any one of claims 2-8, wherein the AML is unfit ND AML.

10. The method of any one of claims 1, 2, and 4-9, wherein the ND AML is a FLT3 TKD, IDH1^{mut}, NMP1^{mut} and/or K/NAS^{mut} AML.

11. The method of any one of claims 1-3 and 5-10, wherein the administration of the combination therapy achieves a minimal residual disease (MRD)-negative status in 2 months or less.

12. The method of any one of claims 1-11, wherein the method further comprises administering a hematopoietic stem cell transplant (HSCT) to the subject.

13. The method of claim 12, wherein the HSCT is allogeneic.

14. The method of claim 12, wherein the HSCT is autologous.

15. The method of any one of claims 12-14, wherein the HSCT is administered about 4 to about 8 weeks after the administration of pivekimab sunirine.

16. A method of preparing a subject with acute myeloid leukemia (AML) for a hematopoietic stem cell transplant (HSCT), the method comprising administering to the subject a combination therapy comprising (i) pivekimab sunirine, (ii) a BCL-2 inhibitor, and (iii) a hypomethylating agent.

17. A method for treating an acute myeloid leukemia (AML) in a subject comprising (i) administering to the subject a combination therapy comprising (i) pivekimab sunirine, (ii) a BCL-2 inhibitor, and (iii) a hypomethylating agent and (ii) subsequently administering a hematopoietic stem cell transplant (HSCT) to the subject.

18. A method for treating an acute myeloid leukemia (AML) in a subject comprising administering a hematopoietic stem cell transplant (HSCT) to the subject, wherein the subject has previously been treated with pivekimab sunirine.

19. The method of any one of claims 16-18, wherein the HSCT is allogeneic.

20. The method of any one of claims 16-18, wherein the HSCT is autologous.

21. The method of any one of claims 16-20, wherein the HSCT is administered about 4 to about 8 weeks after the administration of pivekimab sunirine.

22. The method of any one of claims 16-21, wherein the administration achieves a minimal residual disease (MRD)-negative status.

23. The method of any one of claims 16-22, wherein the AML is a FLT3 TKD, IDH1^{mut}, NMP1^{mut}, and/or K/NAS^{mut} AML.

24. The method of any one of claims 16-23, wherein the pivekimab sunirine is administered once every three weeks (Q3W).

25. The method of any one of claims 1-24, wherein the administration of the combination therapy results in survival for at least 3 months.

26. The method of any one of claims 1-24, wherein the administration of the combination therapy results in survival for at least 6 months.

27. The method of any one of claims 1-26, wherein the administration of the combination therapy achieves undetectable disease.

28. The method of any one of claims 1-27, wherein the administration of the combination therapy results in a pivekimab sunirine Cmax of about 400 to about 600 ng/mL after a single administration of the pivekimab sunirine and/or a pivekimab sunirine Cmax of about 550 to 700 ng/mL after three administrations of the pivekimab sunirine.

29. The method of any one of claims 1-28, wherein the administration of the combination therapy results in a pivekimab sunirine AUC_{INF} of about 4,500 to about 5,000 h*ng/mL after a single administration of the pivekimab sunirine and/or a pivekimab sunirine AUC_{INF} of about 4,750 to about 5,250 h*ng/mL after three administrations of the pivekimab sunirine.

30. The method of any one of claims 1-29, wherein the administration of the combination therapy results in no more than 30% CD123 receptor availability 4 hours after administration of the pivekimab sunirine.

31. The method of any one of claims 1-30, wherein the AML is a TP53^{WT} AML.

32. The method of claim 31, wherein the AML is a K/NRAS^{WT} AML with no FLT3-ITD.

33. The method of claim 31, wherein the AML is a FLT3-ITD or K/NRAS^{mut} AML.

34. The method of any one of claims 1-30, wherein the AML is a TP53^{mut} AML.

35. The method of any one of claims 1-30, wherein the AML is a FLT3 ITD or TKD, IDH1^{mut}, IDH2^{mut}, NPM1^{mut}, and/or K/NRAS^{mut} AML.

36. The method of any one of claims 1-30, wherein the subject has a baseline mutation in RUNX1, ASXL1, or TP53.

37. The method of any one of claims 1-36, wherein the AML has a favorable ELN 2017 risk.

38. The method of any one of claims 1-36, wherein the AML has an intermediate ELN 2017 risk.

39. The method of any one of claims 1-36, wherein the AML has an adverse ELN 2017 risk.

40. The method of any one of claims 1-39, wherein the subject was previously treated with an anti-CD123 therapy, optionally wherein the anti-CD123 therapy was tagraxofusp (SL-401).

41. The method of any one of claims 1-39, wherein the subject has not received tagraxofusp (SL-401) prior to the administration of the combination therapy.

42. The method of any one of claims 1-41, wherein the AML expresses multidrug resistance 1 (MDR1).

43. The method of any one of claims 1-42, wherein the AML expresses P-glycoprotein (P-gp).

44. The method of any one of claims 1-43, wherein the subject has an absolute neutrophil count of greater than 500/L (microliter).

45. The method of any one of claims 2, 3-8, and 10-44, wherein the AML is unfit AML.

46. The method of any one of claims 2, 3-8, and 10-44, wherein the AML is fit AML.

47. The method of any one of claims 1-46, wherein the cancer has previously been treated with venetoclax.

48. The method of any one of claims 1-46, wherein the cancer has not previously been treated with venetoclax.

49. The method of any one of claims 1-48, wherein the cancer has previously been treated with a hypomethylating agent.

50. The method of any one of claims 1-48, wherein the cancer has not previously been treated with a hypomethylating agent.

51. The method of any one of claims 1-50, wherein the subject received a stem cell transplant prior to the administration of the combination therapy.

52. The method of claim 51, wherein the stem cell transplant was received at least 120 days prior to the administration.

53. The method of claim 51 or 52, wherein the previous stem cell transplant was allogeneic.

54. The method of claim 51 or 52, wherein the previous stem cell transplant was autologous.

55. The method of any one of claims 16-54, wherein the AML is a relapsed and/or refractory AML.

56. The method of claim 55, wherein the AML is a relapsed AML.

57. The method of claim 55, wherein the AML is a refractory AML.

58. The method of any one of claims 16-57, wherein the subject received at least one prior line of therapy, at least two prior lines of therapy, or at least three prior lines of therapy.

59. The method of any one of claims 1-15, and 25-54 wherein the administration of the combination therapy is a frontline therapy.

60. The method of any one of claims 1-59, wherein the AML is de novo AML.

61. The method of any one of claims 1-59, wherein the subject has a prior or concomitant hematologic malignancy (PCHM).

62. The method of claim 61, wherein the PCHM does not require immediate therapy.

63. The method of claim 61 or 62, wherein the PCHM is in remission and the subject has completed all therapies for the PCHM at least 6 months prior to the administration of the combination therapy.

64. The method of any one of claims 1-63, wherein the subject has not received immunosuppressive treatment for at least 14 days prior to the administration of the combination therapy.

65. The method of any one of claims 1-64, wherein the subject has not received a systemic anti-cancer therapy for at least 14 days prior to the administration of the combination therapy.

66. The method of any one of claims 1-64, wherein the subject has received a local therapy at least 14 days prior to the administration of the combination therapy.

67. The method of claim 66, wherein the therapy was radiotherapy.

68. The method of any one of claim 1-67, wherein the method further comprises administration of ursodeoxycholic acid.

69. The method of any one of claims 1-68, wherein the subject has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

70. The method of any one of claims 1-69, wherein the pivekimab sunirine is administered intravenously.

71. The method of any one of claims 1-70, wherein the pivekimab sunirine is administered by a controlled rate of infusion.

72. The method of any one of claims 1-71, wherein the pivekimab sunirine is administered in an infusion having a duration of no more than 30 minutes.

73. The method of any one of claims 1-72, wherein the pivekimab sunirine is administered in an infusion having a duration of about 15 to 30 minutes.

74. The method of any one of claims 71-73, wherein the controlled rate of infusion is from about 0.8 m/min to about 1.7 mL/min.

75. The method of any one of claims 71-73, wherein the controlled rate of infusion is about 0.8 mL/min (about 50 mL/hour or about 0.165 mg/min) for the first 30 minutes.

76. The method of any one of claims 71-74, wherein the controlled rate of infusion is increased to about 1.7 mL/min (about 100 mL/hour or about 0.33 mg/min).

77. The method of any one of claims 1-76, wherein the pivekimab sunirine is administered at a dose of about 0.045 mg/kg.

78. The method of any one of claims 1-15, wherein the pivekimab sunirine is administered once every 4 weeks (Q4W).

79. The method of any one of claims 1-15 or 78, wherein the BCL-2 inhibitor is administered daily for at least 14 days of a 28-day cycle.

80. The method of any one of claims 1-15 or 78, wherein the BCL-2 inhibitor is administered daily for up to 28 days of a 28-day cycle.

81. The method of any one of claims 1-15 or 78-80, wherein the BCL-2 inhibitor is administered at a dose of up to about 400 mg.

82. The method of any one of claims 1-15 or 78-81, wherein the BCL-2 inhibitor is administered orally.

83. The method of any one of claims 1-15 or 78-82, wherein the BCL-2 inhibitor is venetoclax.

84. The method of any one of claims 1-15 or 78-83, wherein the hypomethylating agent is administered daily for days 1-7 of a 28-day cycle.

85. The method of any one of claims 1-15 or 78-84, wherein the hypomethylating agent is administered at a dose of about 75 mg/m².

86. The method of any one of claims 1-15 or 78-85, wherein the hypomethylating agent is administered subcutaneously or intravenously.

87. The method of any one of claims 1-15 or 78-86, wherein the hypomethylating agent is azacitidine.

88. The method of any one of claims 1-15 or 78-86, wherein the hypomethylating agent is decitabine.

89. The method of any one of claims 1-15 or 78-88, wherein the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for at least 14 days.

90. The method of any one of claims 1-15 or 78-88, wherein the pivekimab sunirine is administered at a dose of 0.045 mg/kg intravenously on day 7; the hypomethylating agent is azacitidine and it is administered at a dose of 75 mg/m² subcutaneously or intravenously on days 1-7, and the BCL-2 inhibitor is venetoclax and it is administered at a dose of 400 mg orally for up to 28 days.

91. The method of any one of claims 1-90, wherein the administration is for one cycle.

92. The method of any one of claims 1-90, wherein the administration is for more than one cycle, optionally wherein the administration is for at least 2 cycles, at least 3 cycles, at least 4 cycles, at least 5 cycles, at least 6 cycles, at least 7 cycles, at least 8 cycles, at least 9 cycles, or at least 10 cycles or wherein the administration is for about 2-4 cycles, about 2-6 cycles, about 2-8 cycles, about 2-10 cycles, or about 2-12 cycles.

93. The method of any one of claims 1-92, wherein the method further comprises administering a reduced dose of the pivekimab sunirine after a dose-limiting toxicity has occurred in the subject and has been reduced to baseline or ≤Grade 2.

94. The method of any one of claims 1-93, wherein the AML is CD123-positive.

95. The method of any one of claims 1-94, wherein CD123 has been detected in a sample obtained from the AML prior to the administration of the combination therapy, optionally wherein the CD123 was detected using flow cytometry or immunohistochemistry.

96. The method of any one of claims 1-95, further comprising detecting CD123 in a sample obtained from the AML prior to the administration of the combination therapy.

97. The method of any one of claims 1-96, wherein at least 80% of cells in the AML express CD123.

98. The method of any one of claims 1-97, wherein CD123 has been detected in at least 80% of cells in a sample obtained from the AML prior to the administration of the combination therapy.

99. The method of any one of claims 1-98, further comprising detecting CD123 in at least 80% of cells in a sample obtained from the AML prior to the administration of the combination therapy.

100. The method of any one of claims 1-99, wherein the subject is not selected based on level of CD123 expression.

101. The method of any one of claims 1-100, wherein the subject has been pretreated with a premedication or corticosteroid prior to administration of the pivekimab sunirine, optionally wherein the premedication or corticosteroid is diphenhydramine, acetaminophen, paracetamol, dexamethasone, or a combination thereof.

102. The method of any one of claims 1-100, further comprising pre-treating the subject with a premedication or corticosteroid prior to administration of the pivekimab sunirine, optionally wherein the premedication or corticosteroid is diphenhydramine, acetaminophen, paracetamol, dexamethasone, or a combination thereof.

103. The method of any one of claims 101-102, wherein the corticosteroid is administered to a patient on the day prior and once, prior to and on the same day as administering an anti-CD123 immunoconjugate, e.g., pivekimab sunirine, to the patient.

104. The method of any one of claims 101-102, wherein the corticosteroid is administered to a patient twice on the day prior and once, prior to and on the same day as administering an anti-CD123 immunoconjugate, e.g., pivekimab sunirine, to the patient.

105. The method of any one of claims 101-104, wherein the corticosteroid is dexamethasone.

106. The method of claim 105, wherein the dexamethasone is administered at a dose of 8 mg or 10 mg.

107. The method of any one of claims 101-106, wherein the subject is further premedicated with an antihistamine and an antipyretic at least 30 minutes prior to dosing with pivekimab sunirine anti-CD123 immunoconjugate.

108. The method of claim 107, wherein the antihistamine is diphenhydramine and the antipyretic is acetaminophen or paracetamol.

109. The method of claim 108, wherein diphenhydramine is administered intravenously at a dose of 25 mg to 50 mg; and (i) acetaminophen is administered orally or intravenously at a dose of 325 mg to 650 mg or (ii) paracetamol is administered orally or intravenously at a dose of 500 mg to 1000 mg.

110. A method of treating an unfit newly diagnosed acute myeloid leukemia (ND AML) in a subject comprising administering to the subject (i) 0.045 mg/kg pivekimab sunirine, (ii) venetoclax, and (iii) azacitidine, wherein the administration achieves a complete response (CR), CR (clinical) with partial hematological recovery (CRh), CR with incomplete platelet recovery (CRp), or CR (clinical) with incomplete recovery (CRi) with a duration of at least 3 months.

111. The method of claim 110, wherein the AML is a FLT3 TKD, IDH1^{mut}, NMP1^{mut}, and/or K/NAS^{mut} AML.

112. The method of claim 110 or 111, wherein the pivekimab sunirine is administered on day 7 of a 28 day cycle; the is azacitidine and it is administered at a dose of 75 mg/m² on days 1-7 of the cycle, and the venetoclax is administered at a dose of 400 mg for at least 14 days of the cycle.

113. The method of claim 110 or 111, wherein the pivekimab sunirine is administered on day 7 of a 28 day cycle; the is azacitidine and it is administered at a dose of 75 mg/m² on days 1-7 of the cycle, and the venetoclax is administered at a dose of 400 mg for up to 28 days of the cycle.

114. A method of treating relapsed and/or refractory AML in a subject comprising administering to the subject (i) 0.045 mg/kg pivekimab sunirine, (ii) venetoclax, and (iii) azacitidine, wherein the administration achieves a complete response (CR), CR (clinical) with partial hematological recovery (CRh) or CR (clinical) with incomplete recovery (CRi) complete response with a duration of at least 2 months.

115. Use of pivekimab sunirine in the method of any one of claims 1-114 or in the preparation for a medicament for use in the method of any one of claims 1-114.


SEQUENCE LISTING (10 sequences)

SEQ ID NO: 1 - protein, 121 aa, unidentified
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 2 - protein, 108 aa, unidentified
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 3 - protein, 450 aa, unidentified
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 4 - protein, 214 aa, unidentified
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, unidentified
SSIMH

SEQ ID NO: 6 - protein, 17 aa, unidentified
YIKPYNDGTKYNEKFKG

SEQ ID NO: 7 - protein, 12 aa, unidentified
EGGNDYYDTMDY

SEQ ID NO: 8 - protein, 11 aa, unidentified
RASQDINSYLS

SEQ ID NO: 9 - protein, 7 aa, unidentified
RVNRLVD

SEQ ID NO: 10 - protein, 9 aa, unidentified
LQYDAFPYT